(19) United States
(12) Patent Application Publication
(10) Pub. No.: US 2018/0137342 A1
(43) Pub. Date: May 17, 2018

(54) SKIN ANALYZER

(71) Applicant: LARGAN PRECISION CO., LTD., Taichung City (TW)

(72) Inventors: Chen-Yi HUANG, Taichung City (TW); Yi-Chen HSIEH, Taichung City (TW); Yu-Cheng KE, Taichung City (TW); Tsung-Yung HUNG, Taichung City (TW)

(21) Appl. No.: 15/868,121

(22) Filed: Jan. 11, 2018

Related U.S. Application Data
(63) Continuation-in-part of application No. 15/297,223, filed on Oct. 19, 2016.

(30) Foreign Application Priority Data
Jul. 14, 2016 (TW) 105210610
Aug. 2, 2017 (TW) 106211360

(51) Int. Cl. G06K 9/00; A61B 5/00; G06T 7/136; G06T 7/00

(52) U.S. Cl. CPC G06K 9/00255; A61B 5/443; A61B 5/0077; G06T 7/136; H04N 5/2352; A61B 2560/0233; G06T 2207/30088; A61B 5/442; G06T 7/0012

(57) ABSTRACT

A skin analyzer includes a base, an optical imaging system, a flash module, a circuit module, a computing module and a display module. The optical imaging system is disposed on the base for capturing an image of an imaging area. The flash module is disposed on at least one side of the optical imaging system. The circuit module is disposed in the base and electrically connected with the optical imaging system and the flash module. The computing module has a signal transmitting connection with the circuit module. The display module has a signal transmitting connection with the computing module.

### RELATED APPLICATIONS

This application claims priority to Taiwan Application Serial Number 106211360, filed Aug. 2, 2017, and is a Continuation-in-part of U.S. application Ser. No. 15/297,223, filed on Oct. 19, 2016, which claims priority of Taiwan Application Serial Number 105210610, filed Jul. 14, 2016, all of which are herein incorporated by reference.

### BACKGROUND

### Technical Field

The present disclosure relates to a skin analyzer. More particularly, the present disclosure relates to a skin analyzer for suppressing image signal disturbance from overlapping signals.

### Description of Related Art

It's our human nature to appreciate beauty. No matter in aesthetics or from a physical health point of view, everyone wants to have an attractive appearance. The condition of our facial skin is a decisive factor for judging a person's attractiveness. Therefore, an assessment of the facial skin condition of a subject becomes very important now. Conventionally, in the field of cosmetic treatments, the evaluation of the skin condition is done subjectively by naked eyes of a medical practitioner with his or her past experiences. The accuracy of the evaluation is oftentimes debatable and cannot show the whole picture of the subject's condition.

The imaging technology is developing vigorously in recent years and thus an image of the facial skin of a subject can be captured by a high-resolution camera. Then, the skin image information can be digitized by processing the image through an image recognition algorithm for an objective diagnosis. For example, the image recognition algorithm can be an independent component analysis (ICA). The independent component analysis isolates elements like hemoglobin and melanin of the facial skin from the subject into a first independent component and a second independent component, respectively, for determining the current skin condition of the subject. In short, the independent component analysis first captures the facial skin image of the subject with the high-resolution camera. Original image information of red band (R), green band (G) and blue band (B) will be processed by a conversion equation so as to be converted into three independent components. The first independent component is used for determining a distribution of the hemoglobin, and the second independent component is used for determining a distribution of the melanin. However, there are overlapping signal occurrences between different band signals in the original RGB data. One is between the blue band and the green band. The other is between the green band and the red band. Thus, each of the converted independent components cannot effectively present original features of the facial skin of the subject. That is, an accurate image cannot be provided after the conversion.

Moreover, because the size of the conventional skin analyzer is too large to be portable, the subject is required to obtain the evaluation of the skin condition at specialized locations, such as medical institutions or exhibition events. In addition, the user interface of the conventional skin analyzer is too complicated and needs to be operated by a professional specialist, so that the applicability and the universality of the conventional skin analyzer are limited.

Accordingly, a skin analyzer, which can improve the quality of the converted image as well as be more portable and easy to operate, is needed in the market.

### SUMMARY

According to one aspect of the present disclosure, a skin analyzer includes a base, an optical imaging system, at least one flash module, a circuit module, a computing module and a display module. The optical imaging system is disposed on the base and includes an imaging polarizer, a band-stop filter set and an imaging module. The flash module is disposed on at least one side of the optical imaging system, wherein the flash module includes a flash polarizer, a flash lamp and a flash activation circuit. The circuit module is disposed in the base and electrically connected with the optical imaging system and the flash module, wherein the circuit module includes a power control circuit, a data transmission circuit and a signal synchronization circuit. The computing module has a signal transmitting connection with the circuit module. The display module has a signal transmitting connection with the computing module.

The skin analyzer is a substantially rectangular parallelepiped and has a long-side length of the skin analyzer and a short-side length of the skin analyzer. When the long-side length of the skin analyzer is Ls, and the short-side length of the skin analyzer is Ws, the following condition can be satisfied:

0 cm<Ws<Ls<30 cm.

According to another aspect of the present disclosure, a skin analyzer includes a base, an optical imaging system, at least one flash module, a circuit module, a computing module and a display module. The optical imaging system is disposed on the base and includes an imaging module. The flash module is disposed on at least one side of the optical imaging system, wherein the flash module includes a red-light flash, a green-light flash, a blue-light flash and a flash activation circuit. The circuit module is disposed in the base and electrically connected with the optical imaging system and the flash module, wherein the circuit module includes a power control circuit, a data transmission circuit and a signal synchronization circuit. The computing module has a signal transmitting connection with the circuit module. The display module has a signal transmitting connection with the computing module. The skin analyzer is a substantially rectangular parallelepiped and has a long-side length of the skin analyzer and a short-side length of the skin analyzer. When the long-side length of the skin analyzer is Ls, and the short-side length of the skin analyzer is Ws, the following condition can be satisfied:

0<Ws<Ls<30 cm.

### BRIEF DESCRIPTION OF THE DRAWINGS

The present disclosure can be further understood by reading the following detailed description of the embodiment, with reference made to the accompanying drawings as follows:

FIG. 1 is a schematic view of a skin analyzer according to one embodiment of the present disclosure;

FIG. 2A is a schematic view of an optical imaging system of FIG. 1;

FIG. 2B is another schematic view of the optical imaging system of FIG. 1;

FIG. 3 is a schematic view of a skin analyzer according to another embodiment of the present disclosure;

FIG. 4A is a schematic view of the optical imaging system of FIG. 3;

FIG. 4B is a detailed schematic view of the optical imaging system of FIG. 4A;

FIG. 4C is another schematic view of the optical imaging system of FIG. 4B;

FIG. 4D is another schematic view of the optical imaging system of FIG. 3;

FIG. 5 is a schematic view of the flash module of FIG. 3;

FIG. 6A is a front view of a skin analyzer according to a first example of the present disclosure;

FIG. 6B is a three-dimensional view of the skin analyzer according to the first example of the present disclosure;

FIG. 6C is a right-side view of the skin analyzer according to the first example of the present disclosure;

FIG. 6D is a three-dimensional view of the skin analyzer of FIG. 3B without the configuration of a portable device;

FIG. 6E is a rear side view of the skin analyzer according to the first example of the present disclosure;

FIG. 7A is a drawing showing response curves of an image sensor of a skin analyzer without the configuration of a band-stop filter set;

FIG. 7B is a drawing showing a transmission data of a first band-stop filter of the skin analyzer according to the first example of the present disclosure;

FIG. 7C is a drawing showing a transmission data of a second band-stop filter of the skin analyzer according to the first example of the present disclosure;

FIG. 7D is a drawing showing response curves of an image sensor of the skin analyzer according to the first example of the present disclosure;

FIG. 8A is a three-dimensional view of a skin analyzer according to a second example of the present disclosure;

FIG. 8B is a three-dimensional view of the skin analyzer of FIG. 8A without the configuration of a portable device;

FIG. 8C is a schematic view of a flash module in a folded position of the skin analyzer according to the second example of the present disclosure;

FIG. 9 is a structural schematic view of the flash module of the skin analyzer according to the second example of the present disclosure;

FIG. 10 is schematic view showing an operation status of the skin analyzer according to the first example of the present disclosure;

FIG. 11 is a flow chart of an image analysis process of the skin analyzer according to the first example of the present disclosure;

FIG. 12A is a front view of a skin analyzer according to a third example of the present disclosure;

FIG. 12B is a three-dimensional view of the skin analyzer of FIG. 12A;

FIG. 12C is a front view of the skin analyzer of FIG. 12A without the configuration of a display module;

FIG. 12D is a right-side view of the skin analyzer of FIG. 12A without the configuration of the display module;

FIG. 12E is a rear side view of the skin analyzer of FIG. 12A;

FIG. 12F is a schematic view of a standing angle adjusting apparatus of the skin analyzer of FIG. 12A;

FIG. 13 is a flow chart of an image analysis process of the skin analyzer according to the third example of the present disclosure;

FIG. 14 is a front side view of a skin analyzer according to a fourth example of the present disclosure; and

FIG. 15 is a flow chart of an image analysis process of the skin analyzer according to the fourth example of the present disclosure.

### DETAILED DESCRIPTION

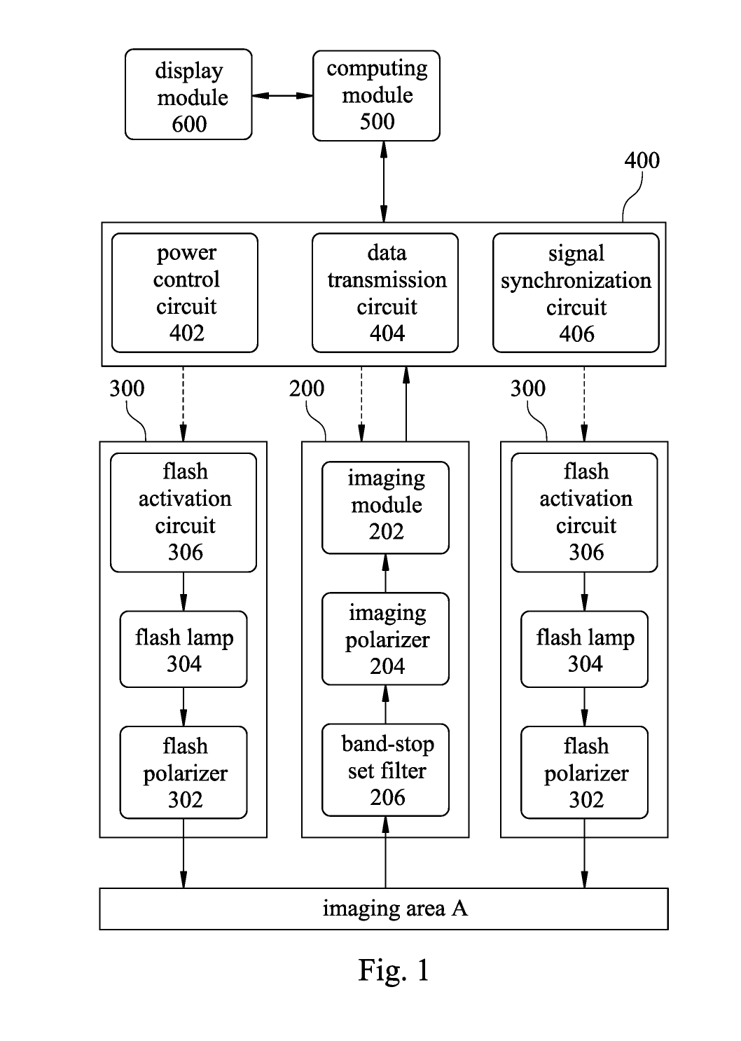

Please refer to FIG. 1. FIG. 1 is a schematic view of a skin analyzer according to one embodiment of the present disclosure. The present disclosure provides a skin analyzer for detecting a skin condition of an imaging area A of a subject (not shown). The skin analyzer includes a base (not shown), an optical imaging system 200, at least a flash module 300, a circuit module 400, a computing module 500 and a display module 600.

Although it is not shown by the figure, the base is provided for supporting other components of the skin analyzer. The base of the skin analyzer can be a hollow case and made of a plastic material.

The optical imaging system 200 is disposed on the base facing toward the imaging area and includes an imaging module 202, an imaging polarizer 204 and a band-stop filter set 206.

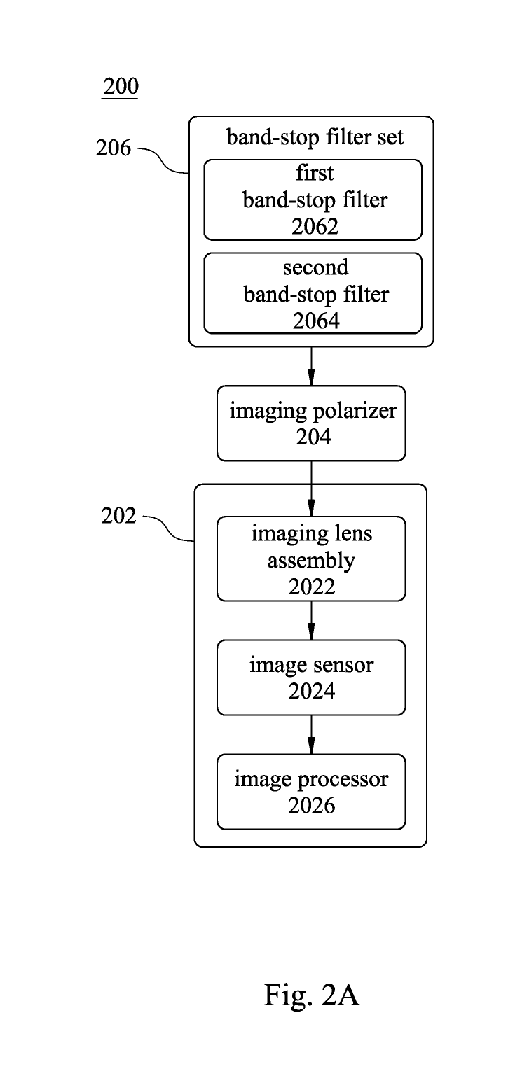

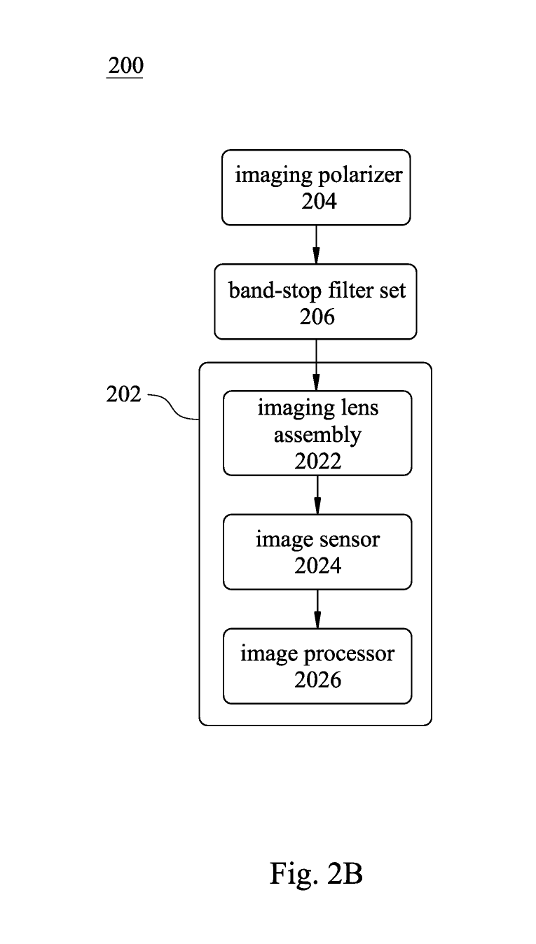

Please refer to FIGS. 2A and 2B. FIG. 2A is a schematic view of the optical imaging system 200 of FIG. 1. FIG. 2B is another schematic view of the optical imaging system 200 of FIG. 1. As shown in FIG. 2A, the imaging module 202 includes an imaging lens assembly 2022, an image sensor 2024 and an image processor 2026. The imaging lens assembly 2022 includes a plurality of lens elements. The number of the lens elements and the configuration of the imaging lens assembly 2022 is not a subject matter in the present disclosure, so that the details of the imaging lens assembly 2022 are not be described herein. The image sensor 2024 can be a charge-coupled device (CCD) or a complementary metal-oxide-semiconductor (CMOS). The image processor 2026 can be a dedicated graphics card or an integrated graphics processor. The image processor 2026 is mainly provided for processing the image retrieved from the optical imaging system 200, and transmitting image information to the computing module 500 via the circuit module 400.

Subsequently, the imaging polarizer 204 is located between the imaging module 202 and the imaging area A. The imaging polarizer 204 can be a linear polarizer, a circular polarizer or an elliptical polarizer.

The band-stop filter set 206 is also located between the imaging module 202 and the imaging area A. Additionally, the imaging polarizer 204 can be located between the band-stop filter set 206 and the imaging module 202 as shown in FIG. 2A. The band-stop filter set 206 also can be located between the imaging polarizer 204 and the imaging module 202 as shown in FIG. 2B.

Furthermore, the band-stop filter set 206 can include one multi-band-stop filter or a plurality of single-band-stop filters. In particular, the band-stop filter set 206 includes three or less single-band-stop filters. Furthermore, the band-stop filter set 206 in the present disclosure is provided for suppressing the overlapping signals of the B-G band and the G-R band. Thus, the band-stop filter set 206 can include a first band-stop filter 2062 and a second band-stop filter 2064 as shown in FIG. 2A. The conditions of the abovementioned filters will be further described in the following embodiments. In addition, the single-band-stop filter can be a notch filter.

Please refer to FIG. 1. The flash module 300 is disposed on at least one side of the optical imaging system 200. The flash module 300 includes a flash polarizer 302, a flash lamp 304 and a flash activation circuit 306. The flash polarizer 302 is located between the flash lamp 304 and the imaging area A, and the flash polarizer 302 can be a linear polarizer, a circular polarizer or an elliptical polarizer. The flash lamp 304 can be a xenon flash lamp or a light-emitting diode (LED). The flash activation circuit 306 can be further divided into a capacitive charging circuit and a signal triggering circuit. When the signal triggering circuit is activated, the capacitive charging circuit starts discharging to allow the flash lamp 304 to perform an ambient light compensation. Therefore, the quality of the image captured by the optical imaging system 200 will be enhanced.

The circuit module 400 can be disposed in the base and connected electrically with the optical imaging system 200 and the flash module 300. The circuit module 400 can include a power control circuit 402, a data transmission circuit 404 and a signal synchronization circuit 406. The power control circuit 402 is provided for controlling circuits and power sources, which may be disposed in the abovementioned elements. The data transmission circuit 404 is provided for transmitting image information, which is retrieved by the optical imaging system 200 and transferred to the computing module 500. The signal synchronization circuit 406 is provided for controlling the optical imaging system 200 and the flash module 300 synchronously. In addition, the data transmission circuit 404 includes a wireless transmission module or a wired transmission module. The wireless transmission module can be a Bluetooth wireless transmission module or an infrared wireless transmission module.

The computing module 500 has a signal transmitting connection with the circuit module 400 for receiving image information through the data transmission circuit 404 of the circuit module 400. The computing module 500 will further compute image information to output an analyzed result of the skin condition. In particular, the computing module 500 is provided to check image information captured by the optical Imaging system 200. Furthermore, the computing module 500 analyzes and computes the abovementioned information to produce the analyzed result of the skin condition. Then, the image and the analyzed result are shown in the display module 600. The computing module 500 can be any modules capable of completing the abovementioned operation, such as a microprocessor, a smart mobile device, a personal computer or a server.

The display module 600 has a transmitting connection with the computing module 500 for receiving and displaying the image and the analyzed result of the skin condition. Furthermore, the display module 600 can display interactive information of a user interface (not shown) to be operated by the subject or the medical practitioner. Then, the display module 600 can display the image and the analyzed result of the skin condition from the computing module 500. Thus, the display module 600 can be a thin film transistor liquid crystal display (TFT-LCD), an active-matrix organic light-emitting diode (AMOLED) or a flexible display.

It is noted that the computing module 500 and the display module 600 can be disposed in different devices in the present disclosure. For example, the computing module 500 and the display module 600 can be another microprocessor, portable device or personal computer. The transmission and reception of the image information between the circuit module 400 and each of the computing module 500 and the display module 600 can be completed through the data transmission circuit 404, which utilizes wireless transmission technologies. However, the computing module 500 and the display module 600 also can be integrated into a portable device or a personal computer. Moreover, the computing module 500 and the display module 600 can be built in the base and cooperated with a display for showing the image and the analyzed result of the skin condition.

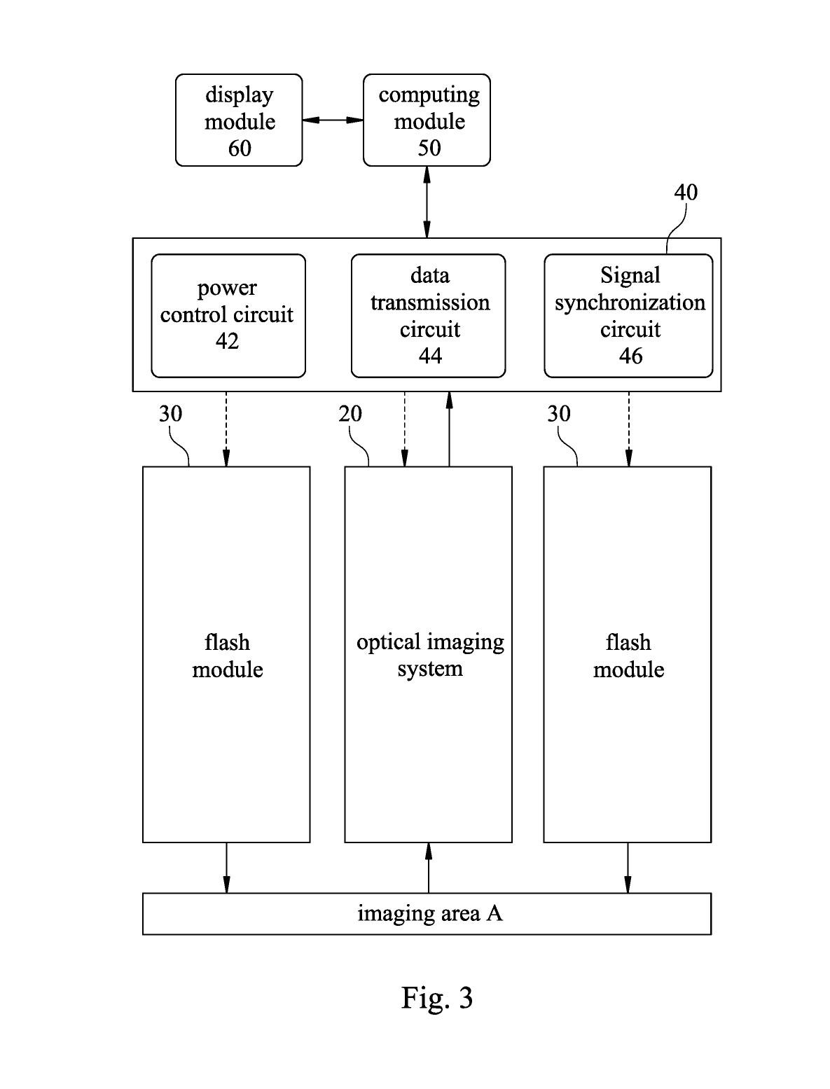

Please refer to FIG. 3, which is a schematic view of a skin analyzer according to another embodiment of the present disclosure. As shown in FIG. 3, the skin analyzer includes a base (not shown), an optical imaging system 20, at least one flash module 30, a circuit module 40, a computing module 50 and a display module 60. The optical imaging system 20 is disposed on the base. The flash module 30 is disposed on at least one side of the optical imaging system 20. The circuit module 40 is disposed in the base and electrically connected with the optical imaging system 20 and the flash module 30. The display module 60 has a signal transmitting connection with the computing module 50. Although it is not shown by FIG. 3, the base of the skin analyzer can be a hollow case which can be provided for supporting other components of the skin analyzer, such as the flash module 30. The skin analyzer can be a substantially rectangular parallelepiped and has a long-side length of the skin analyzer and a short-side length of the skin analyzer. When the long-side length of the skin analyzer is Ls, and the short-side length of the skin analyzer is Ws, the following condition can be satisfied: 0 cm<Ws<Ls<30 cm. Therefore, the size of the skin analyzer can be reduced and it is favorable for higher portability. In particular, when the long-side length of the skin analyzer is Ls, and the short-side length of the skin analyzer is Ws, the following condition can be satisfied: 5 cm<Ls<20 cm. Therefore, the size of the skin analyzer can be further reduced.

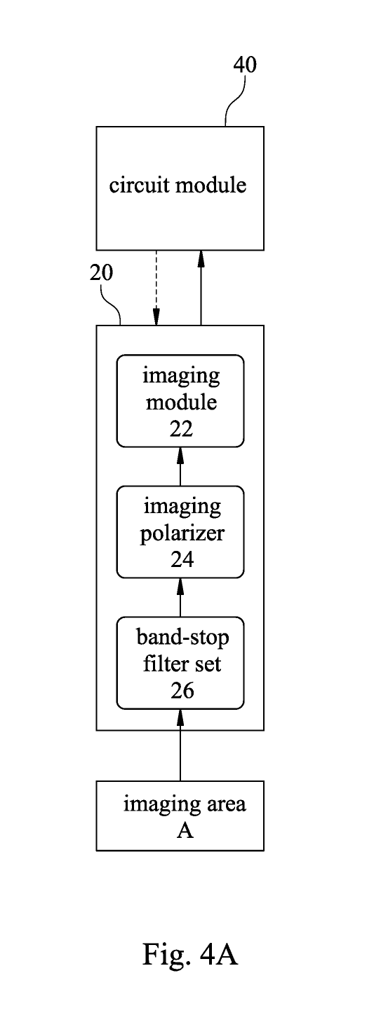

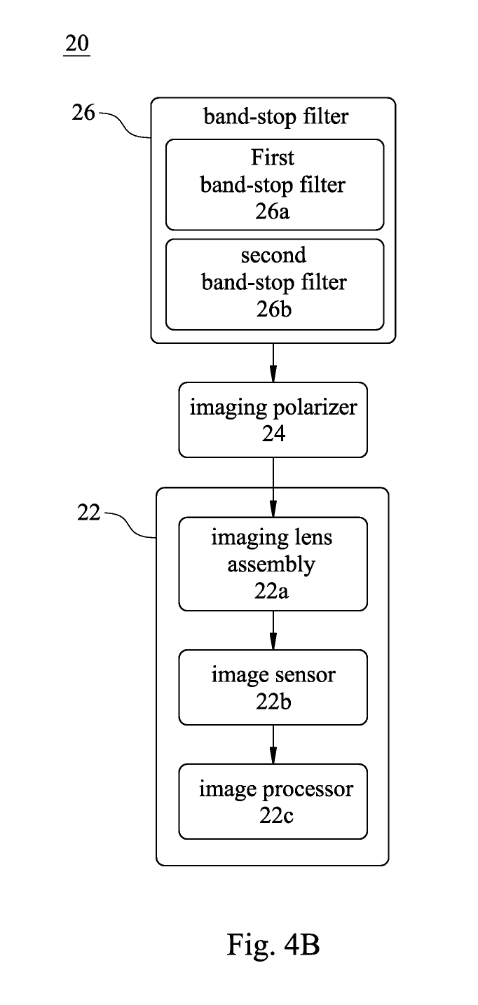

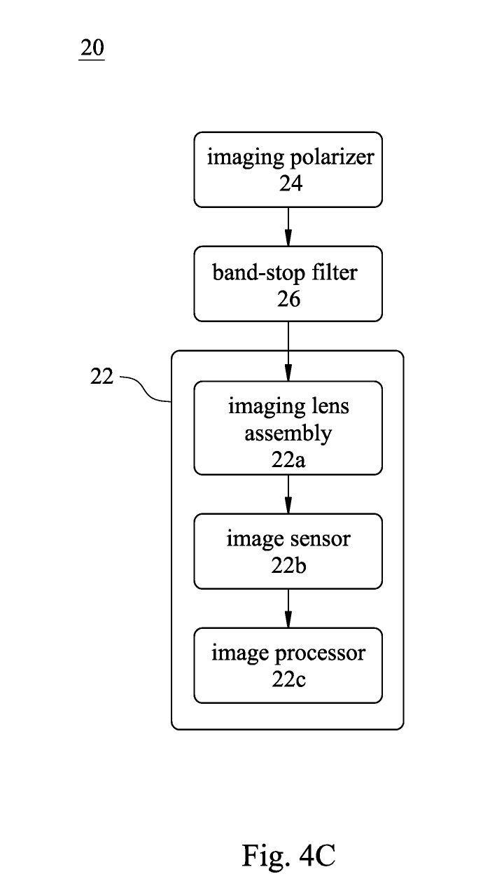

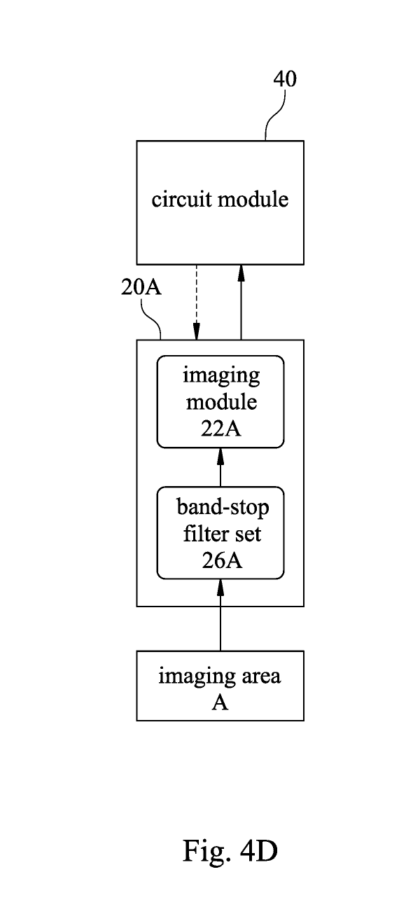

Please refer to FIG. 4A to FIG. 4D. FIG. 4A is a schematic view of the optical imaging system 20 of FIG. 3. FIG. 4B is a detailed schematic view of the optical imaging system 20 of FIG. 4A. FIG. 4C is another schematic view of the optical imaging system 20 of FIG. 4B. FIG. 4D is another schematic view of the optical imaging system 20A of FIG. 3.

As shown in FIG. 4A, the optical imaging system 20 includes an imaging module 22, an imaging polarizer 24 and a band-stop filter set 26. As shown in FIG. 4B, the imaging module 22 particularly includes an imaging lens assembly 22a, an image sensor 22b and an image processor 22c. The imaging lens assembly 22a includes a plurality of lens elements. The number and the configuration of the lens elements of the imaging lens assembly 22a is not a subject matter in the present disclosure, so that the details of the imaging lens assembly 22a are not be described herein. The image sensor 22b can be a charge-coupled device (CCD) or a complementary metal-oxide-semiconductor (CMOS). In particular, a peak quantum efficiency of a red band of the image sensor 22b is less than a peak quantum efficiency of a green band or a blue band of the image sensor 22b. Therefore, the red band noise affecting the identification process of each independent component can be reduced. The image processor 22c can be a dedicated or integrated graphics processor, and the image processor 22c is mainly provided for processing the image retrieved from the optical imaging system 20, and transmitting image information to the computing module 50 via the circuit module 400.

Furthermore, the imaging polarizer 24 is disposed between the imaging module 22 and the band-stop filter set 26. The imaging polarizer 24 can be a linear polarizer, a circular polarizer or an elliptical polarizer, and will not be limited thereto. In addition, as shown in FIG. 4D, when the flash module 30 is to replaced with a full-range flash module (of visible light spectrum, i.e. RGB colors), the optical imaging system 20A includes only an imaging module 22A and a band-stop filter set 26A, and the imaging polarizer can be omitted, as shown in FIG. 4D.

The band-stop filter set 26 is disposed between the imaging polarizer 24 and the imaging area A. However, as shown in FIG. 4C, the band-stop filter set 26 can be disposed between the imaging polarizer 24 and the imaging module 22, but will not be limited thereto. In particular, a full width at a half maximum of a filter region of the band-stop filter set 26 can be less than 40 nm. Therefore, the excessive exclusion of signal information can be avoided and the independent components of the signals can be accurately translated. In addition, when the image information is processed by the independent component analysis, the original image information of red band (R), green band (G) and blue band (B) will be processed by a transform function so as to be converted into three independent components. At this time, the first independent component is used for determining a distribution of the hemoglobin, and the second independent component is used for determining a distribution of the melanin. However, there are overlapping signal occurrences between different band signals in the original RGB data. One is between the blue band and the green band. The other is between the green band and the red band. Thus, each of the converted independent components cannot effectively present original features of the facial skin of the subject. Therefore, as shown in FIG. 4B, the band-stop filter set 26 can include a first band-stop filter 26a and a second band-stop filter 26b, wherein the first band-stop filter 26a is a blue-green filter, the second band-stop filter 26b is a green-red filter. When an upper limit wavelength of a band-stop of the first band-stop filter 26a is WL1, and a lower limit wavelength of a band-stop of the second band-stop filter 26b is WL2, the following condition can be satisfied: 70 nm<WL2−WL1<100 nm. Therefore, the overlapping signals of the B-G band (blue-green) and the G-R band (green-red) can be efficiently adjusted, and the features of the image will not be hindered by the overlapping signals so as to improve the quality of the image after the conversion of the independent component analysis.

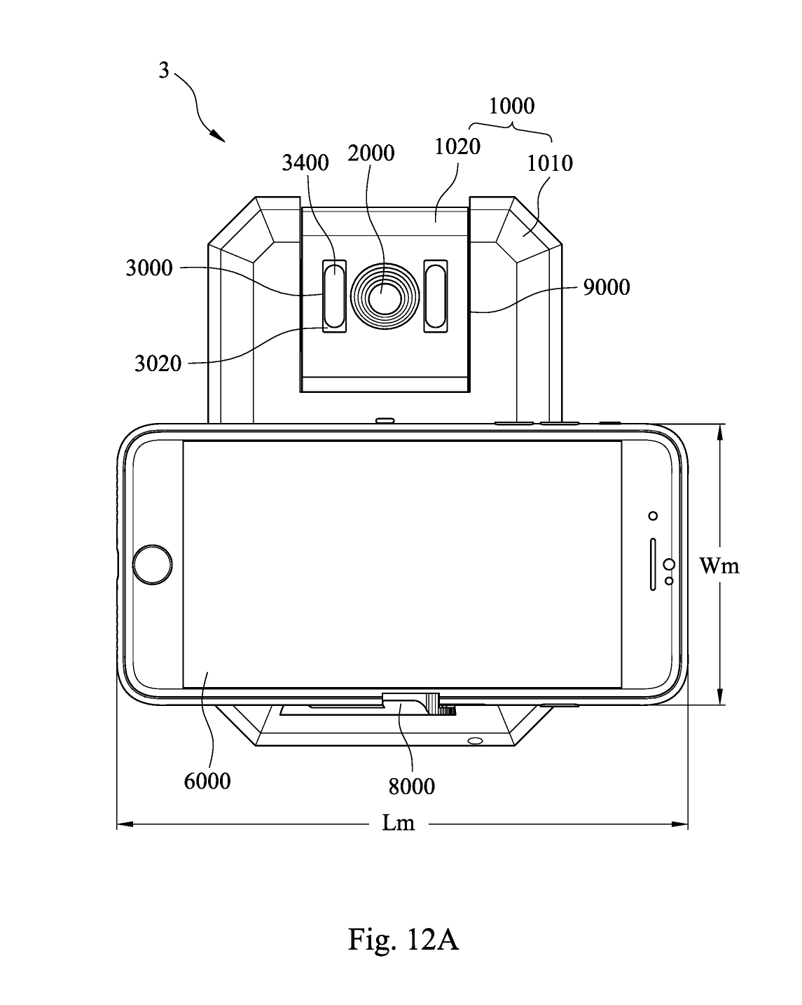

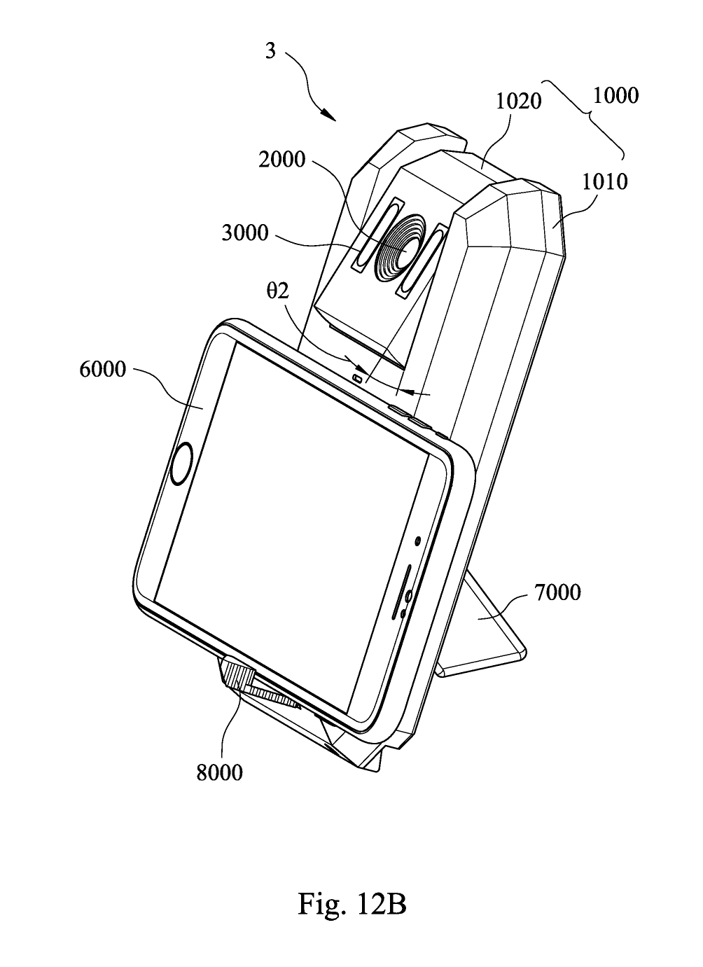

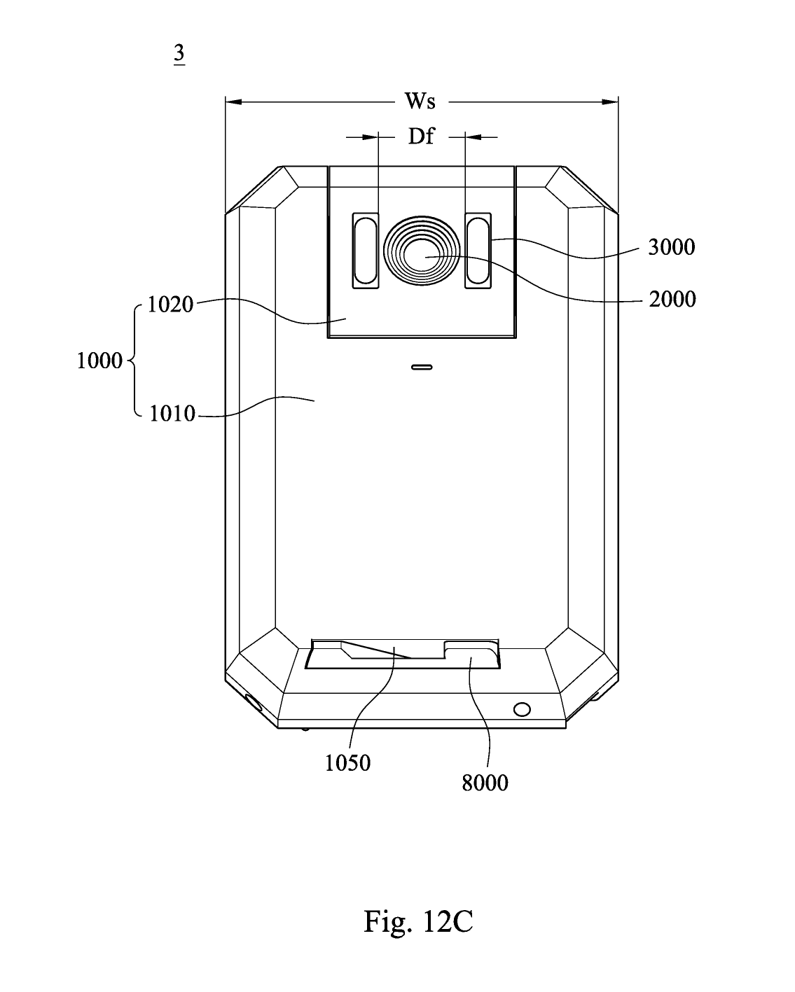

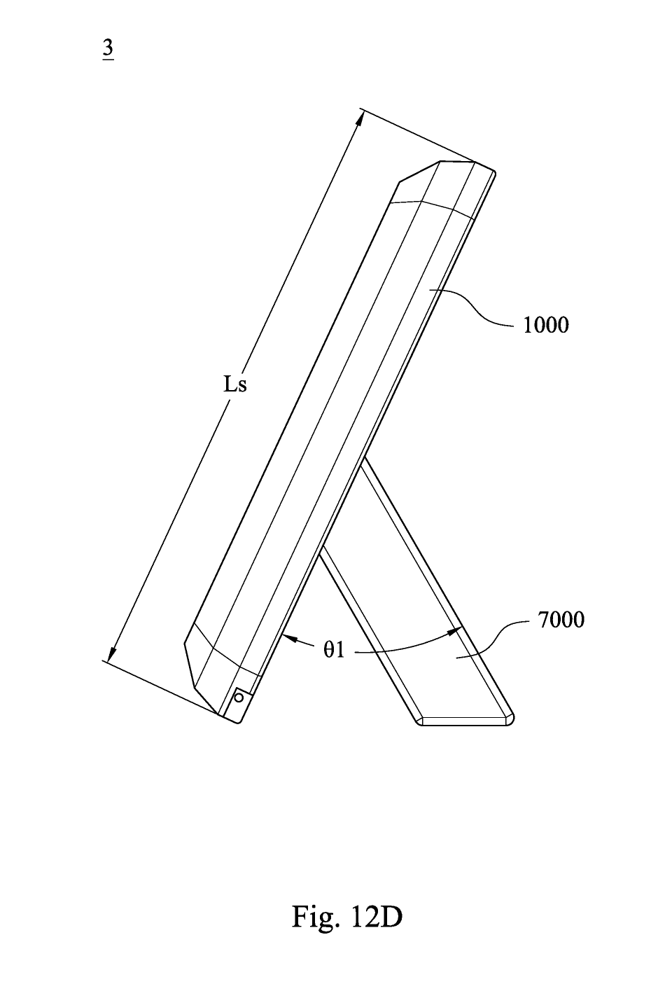

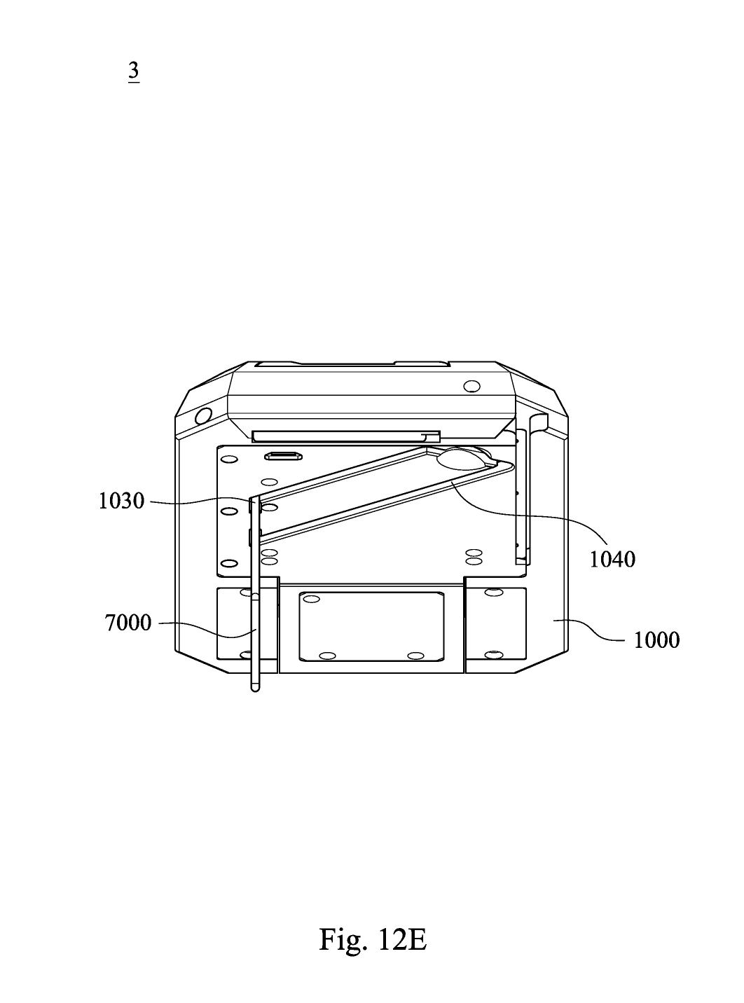

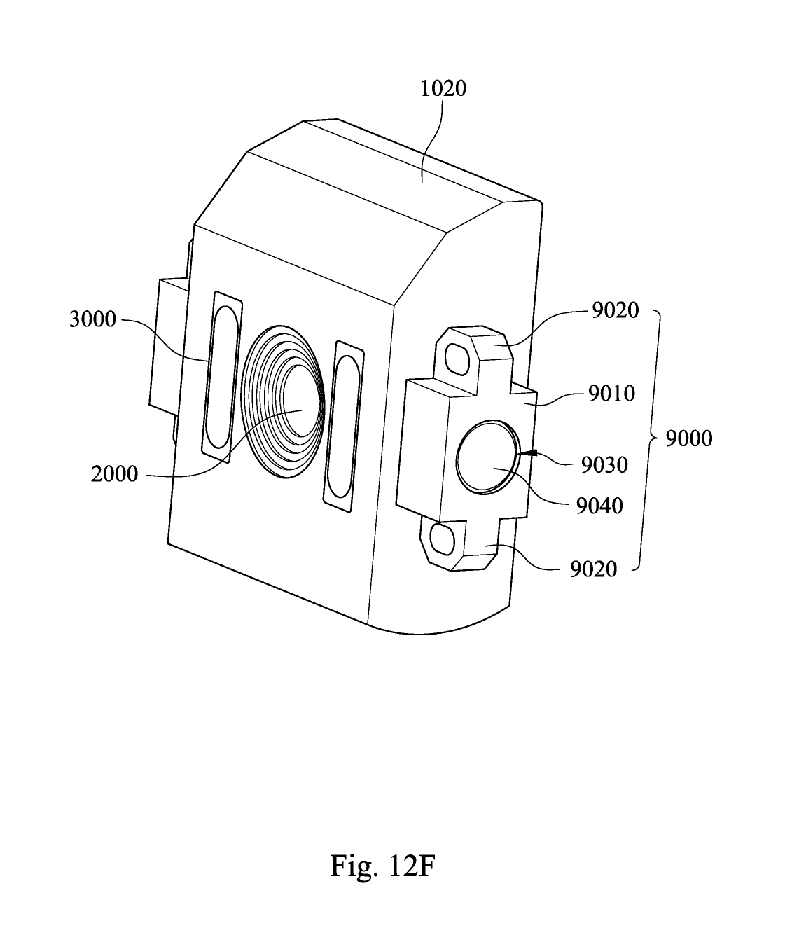

Furthermore, the skin analyzer can include a foldable stand, similar to another foldable stand 7000 shown in FIG. 12B. Therefore, the size of the skin analyzer can be reduced and it is favorable for storage, as well as making the skin analyzer easily accessible on the go. In addition, corresponding to the foldable stand, the base can further include a standing angle adjusting apparatus which is connected with the foldable stand. Therefore, a proper usage angle of the skin analyzer can be obtained by the adjustment of the standing angle adjusting apparatus.

The display module 60 is for receiving and displaying the aforementioned image information as well as the analyzed results. Thus, the display module 60 can be a portable device, that is, the skin analyzer of the present disclosure can be used along with the existing portable device and providing an intuitive and easy-to-operate platform for the subject, and the image capture area of the skin analyzer can be confirmed directly by the subject from the display of the portable device. Furthermore, the skin analyzer further includes a portable device bracket disposed on a side of the skin analyzer facing toward the subject, the portable device is disposed in the portable device bracket, and the base can include a bracket storage apparatus correspondingly. Therefore, the portable device can be removed from the portable device bracket, and then the portable device bracket can be housed in the bracket storage apparatus, so that the goals of reducing the usage space and enhancing the portability of the skin analyzer can be further achieved. In particular, the portable device can be a substantially rectangular parallelepiped and have a long-side length of the portable device and a short-side length of the portable device, wherein the long-side length of the portable device and the short-side length of the skin analyzer are disposed in parallel, and the short-side length of the portable device and the long-side length of the skin analyzer are disposed in parallel. Therefore, the portable device can be stably placed in landscape orientation for the subject while operating the skin analyzer.

In addition, the skin analyzer of the present disclosure can further include a view angle adjusting apparatus. Therefore, the optical imaging system 20 can be rotated upward or downward. In particular, the view angle adjusting apparatus is for rotating the optical imaging system 20 upward or downward of a certain angle, and the angle is less than or equal to 45 degrees. Therefore, the subject can adjust the imaging orientation of the optical imaging system 20 via the view angle adjusting apparatus and confirm directly whether the area of interest falls within the image capture range of the optical imaging system 20 from the screen of the portable device.

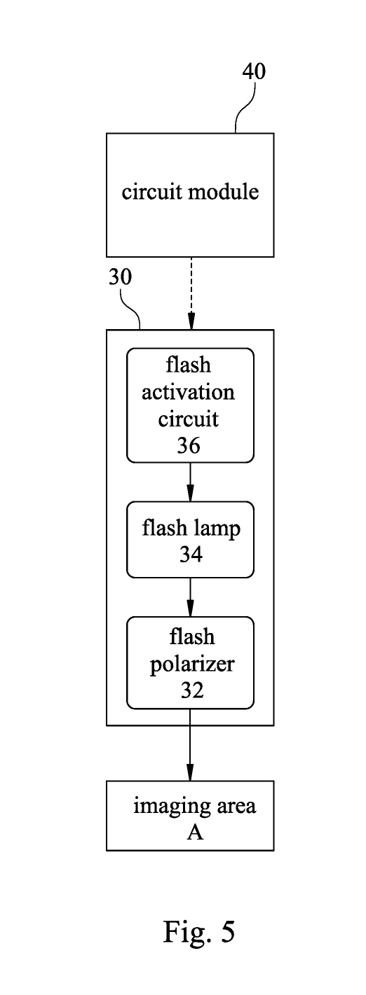

Please refer to FIG. 3 and FIG. 5 simultaneously. FIG. 5 is a schematic view of the flash module 30 of FIG. 3. As shown in FIG. 5, the flash module 30 can include a flash polarizer 32, a flash lamp 34 and a flash activation circuit 36. The flash polarizer 32 is disposed between the flash lamp 34 and the imaging area A. The flash polarizer 32 can be a linear polarizer, a circular polarizer or an elliptical polarizer, and will not be limited thereto.

There can be two of the flash module 30, and the two flash modules 30 are disposed at two sides of the optical imaging system 20, respectively. Therefore, a sufficient light source can be provided by the symmetrical disposition of the two flash modules 30, so that the quality of the image captured by the optical imaging system 20 will be enhanced. In detail, when a distance between the two flash modules 30 is Df, and the short-side length of the skin analyzer is Ws, the following condition can be satisfied: 0.1<Df/Ws<0.7. Therefore, when the distance between the two flash modules 30 is miniaturized, the width of the skin analyzer can be proper for compactness without compromising the image quality.

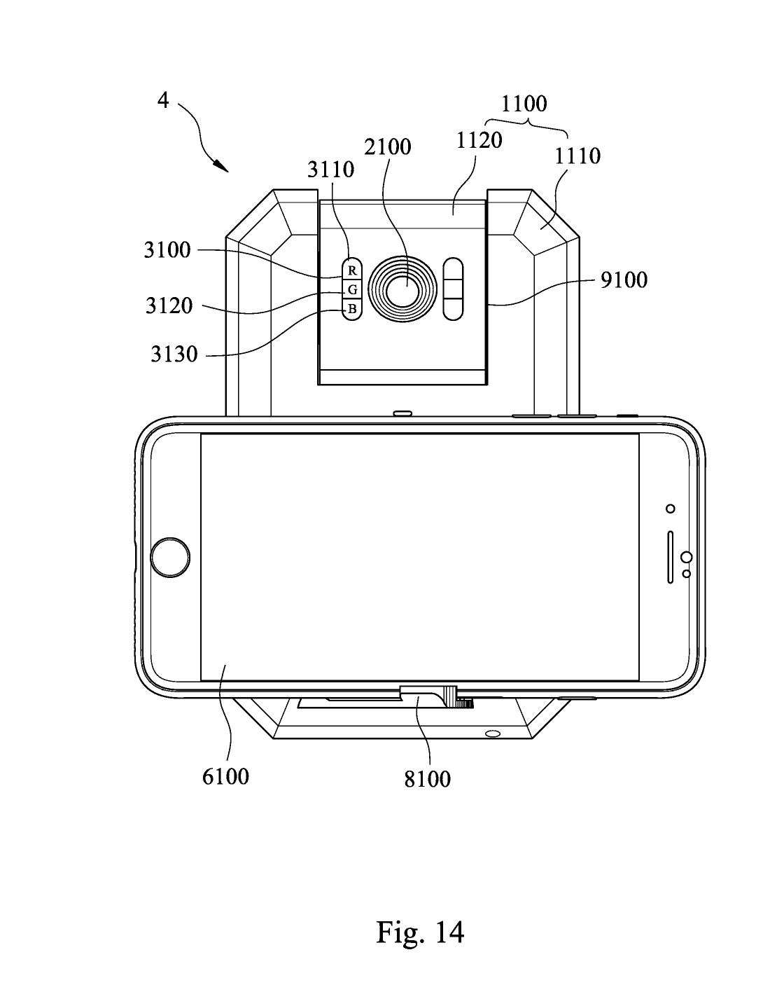

The flash lamp 34 can be a xenon flash lamp or a light-emitting diode (LED). It should be noted that the flash lamp 34 can be replaced with a full band flash module, that is, as shown in FIG. 14, a single flash lamp is replaced with a red flash lamp, a green flash lamp and a blue flash lamp.

The flash activation circuit 36 can be further divided into a capacitive charging circuit and a signal triggering circuit. When the signal triggering circuit is activated, the capacitive charging circuit starts discharging to allow the flash lamp 34 to perform an ambient light compensation. Therefore, the quality of the image captured by the optical imaging system 20 will be enhanced.

Furthermore, as shown in FIG. 3, the circuit module 40 can include a power control circuit 42, a data transmission circuit 44 and a signal synchronization circuit 46. The power control circuit 42 is provided for controlling circuits and power sources, which may be disposed in the abovementioned elements. The data transmission circuit 44 is provided for transmitting image information, which is retrieved by the optical imaging system 20 and transferred to the computing module 50. The signal synchronization circuit 46 is provided for controlling the optical imaging system 20 and the flash module 30 synchronously. In addition, the data transmission circuit 44 includes a wireless transmission module or a wired transmission module. The wireless transmission module can be a Bluetooth wireless transmission module or an infrared wireless transmission module and will not be limited thereto.

The computing module 50 is provided for processing the image information captured by the optical imaging system 20 and further generating an output of analyzed result. Then, the image information and the analyzed result are shown in the display module 60. Thus, the computing module 50 can be any modules capable of completing the abovementioned operation, such as a microprocessor, a smart mobile device, a personal computer, a server, etc.

The display module 60 can display interactive information of a user interface (not shown) and can be operated by the subject. Thus, the display module 60 can display the image and the analyzed result of the skin condition from the computing module 50. Thus, the display module 60 can include a thin film transistor liquid crystal display (TFT-LCD), an active-matrix organic light-emitting diode (AMOLED) or a flexible display.

Furthermore, the computing module 50 and the display module 60 can be integrated into the same device. For example, when the display module 60 is a portable device, the computing module 50 can be an internal microprocessor of the portable device, and the image information can be transmitted by the data transmission circuit 44 of the circuit module 40 via wireless transmission technologies. Alternatively, when the skin analyzer is a portable device, the computing module 50 is the built-in microprocessor of the portable device, and the display module 60 is the built-in display screen of the portable device. In addition, the computing module 50 and the display module 60 can be two different devices. For example, the computing module 50 and the display module 60 can be an independent microprocessor, portable device or personal computer, respectively. The transmission and reception of the image information between the circuit module 40 and the computing module 50, or the display module 60, can be completed through the data transmission circuit 44, which utilizes wireless transmission technologies.

According to the above description of the present disclosure, the following 1st-4th specific examples are provided for further explanation.

### First Example

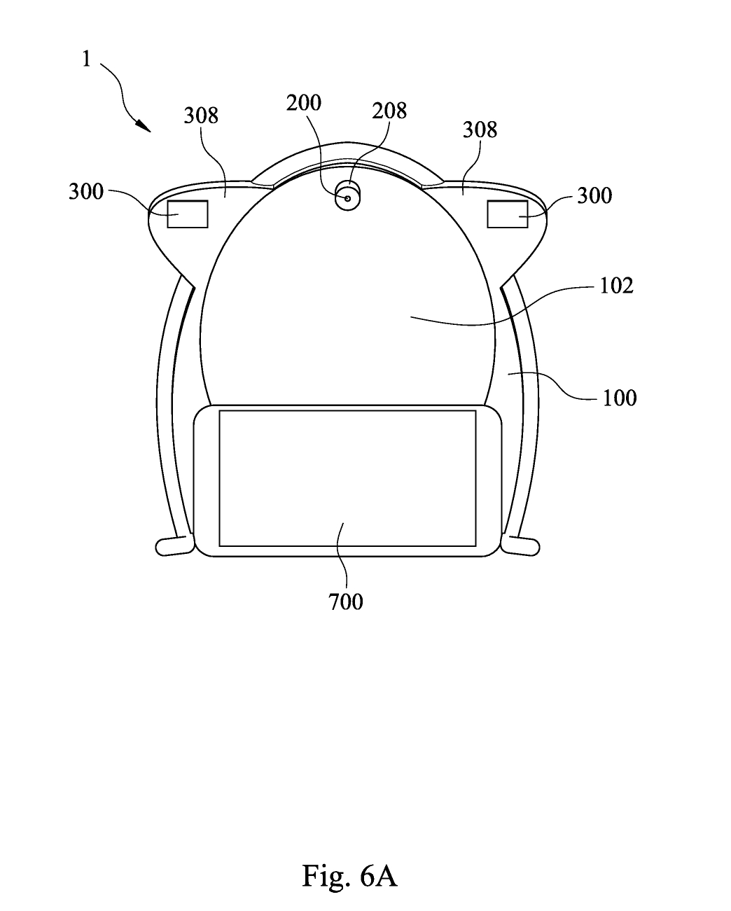

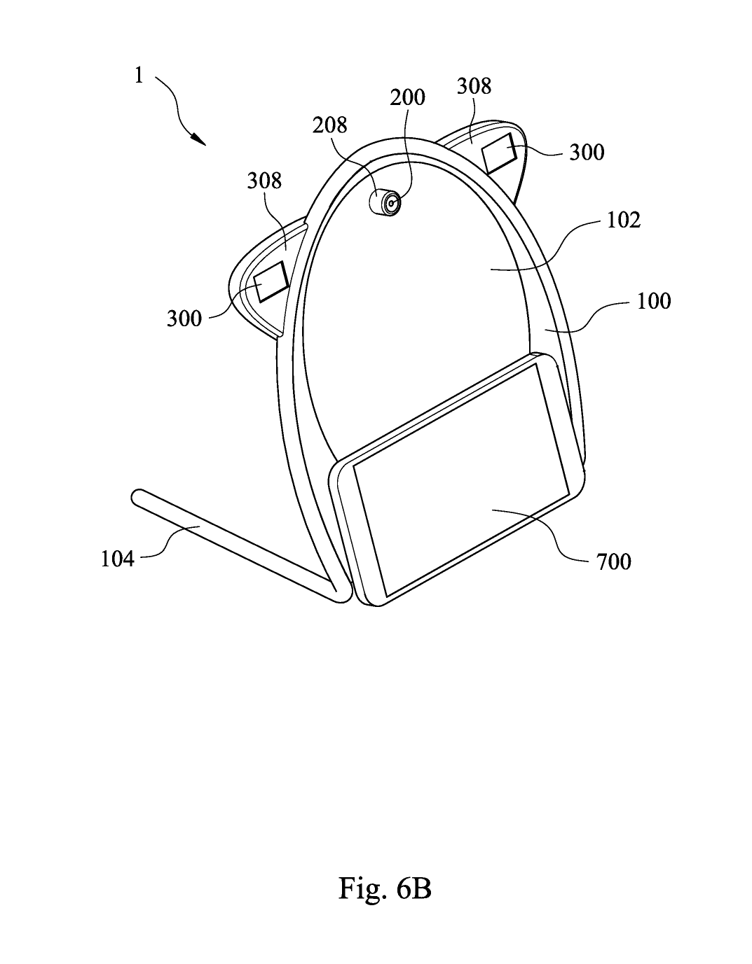

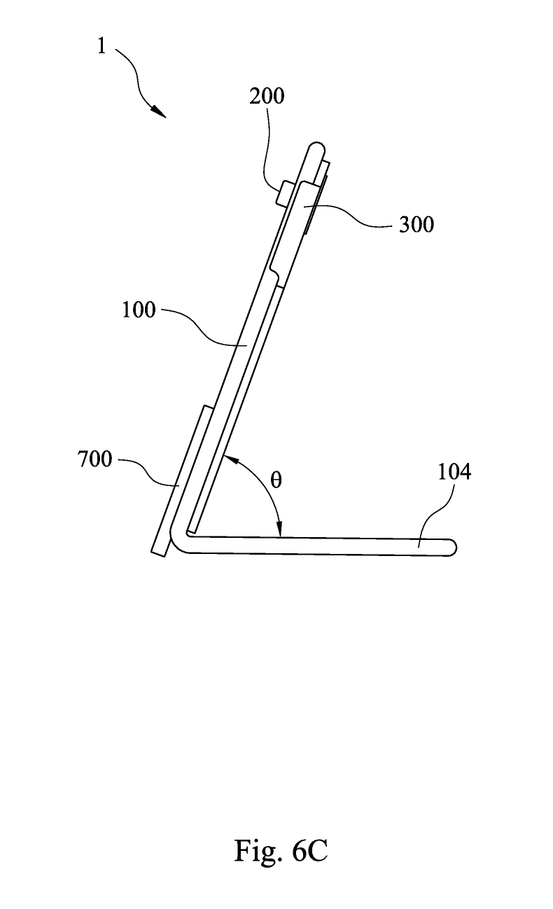

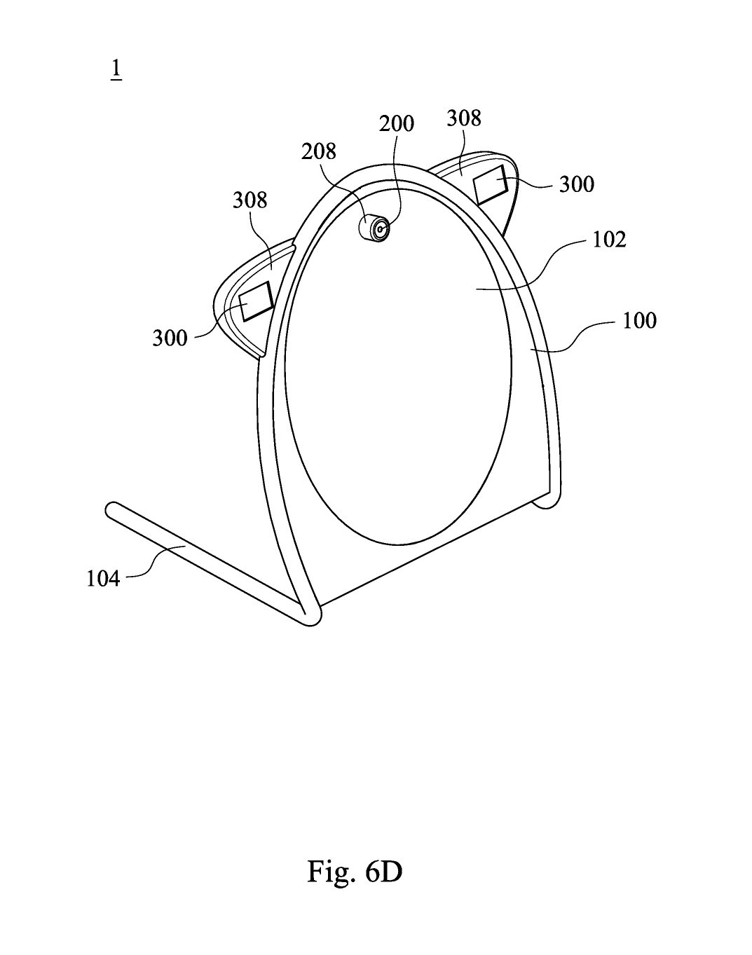

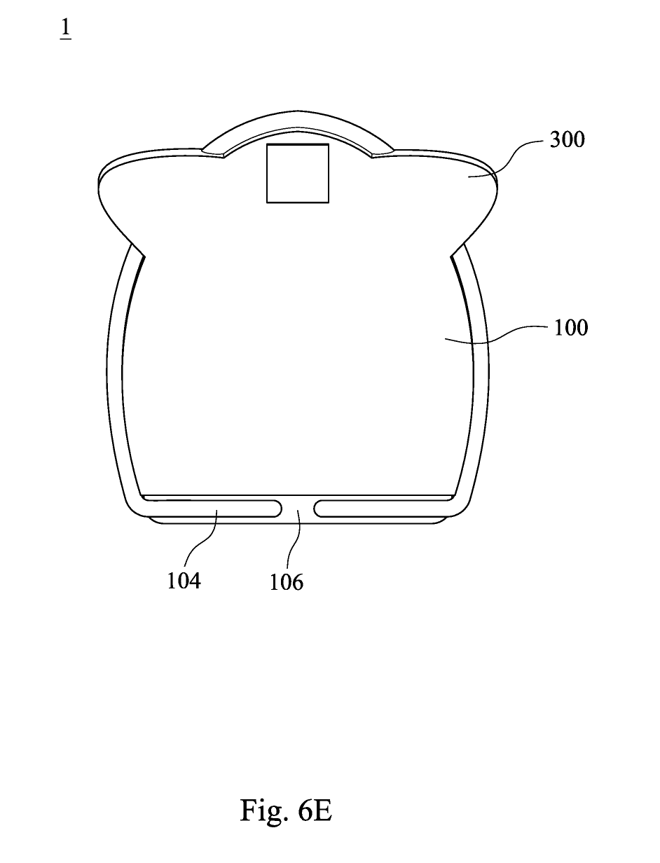

Please refer to FIGS. 6A, 6B, 6C, 6D and 6E. FIG. 6A is a front view of a skin analyzer 1 according to a first example of the present disclosure. FIG. 6B is a three-dimensional view of the skin analyzer 1 according to the first example of the present disclosure. FIG. 6C is a right-side view of the skin analyzer 1 according to the first example of the present disclosure. FIG. 6D is a three-dimensional view of the skin analyzer 1 of FIG. 6B without the configuration of a portable device. FIG. 6E is a rear side view of the skin analyzer 1 according to the first example of the present disclosure. The structure of the skin analyzer 1 according to the first example of the present disclosure is shown in FIG. 1 and FIG. 2A. That is, the skin analyzer of the first example includes a base 100, an optical imaging system 200, at least one flash module 300, a circuit module 400, a computing module 500 and a display module 600. It is noted that the computing module 500 and the display module 600 of the skin analyzer of the first example are built in the portable device 700.

As shown in FIG. 6A and FIG. 6B, the base 100 of the skin analyzer 1 of the first example can be a case with a semi-circle shape. The base 100 has a receiving space (not shown) for other parts if necessary. In addition, the base 100 further includes a mirror portion 102. The mirror portion 102 is disposed at one side of the base 100 while facing the subject. The mirror portion 102 can be utilized to ensure the subject is positioned within the field of view of the optical imaging system 200. As shown in FIG. 6C, the base 100 has a slim shaped structure and the base 100 can further include a stand 104. The stand 104 is connected to a lower portion of the base 100 for supporting the base 100 against a surface plane and has an angle θ with the surface plane. In addition, the stand 104 is rotatable and connected to the base 100 so that the angle θ can be adjusted. Thus, the portable device 700 can be placed against the mirror portion 102 and is held in place by friction thereof.

More particularly, the portable device 700 can be detached from the base 100 when the skin analyzer 1 is not in the use, as shown in FIG. 6D and FIG. 6E. Furthermore, the base 100 further includes a first receiving groove 106. The stand 104 can be rotated and folded into the first receiving groove 106 for compactness of the skin analyzer 1.

In addition, the optical imaging system 200 is disposed at an upper portion of the base 100 and includes a first housing 208 for concealing the abovementioned elements. The first housing 208 is further coupled with the base 100 to prevent elements of the optical imaging system 200 from being affected by the external environmental factors while operating.

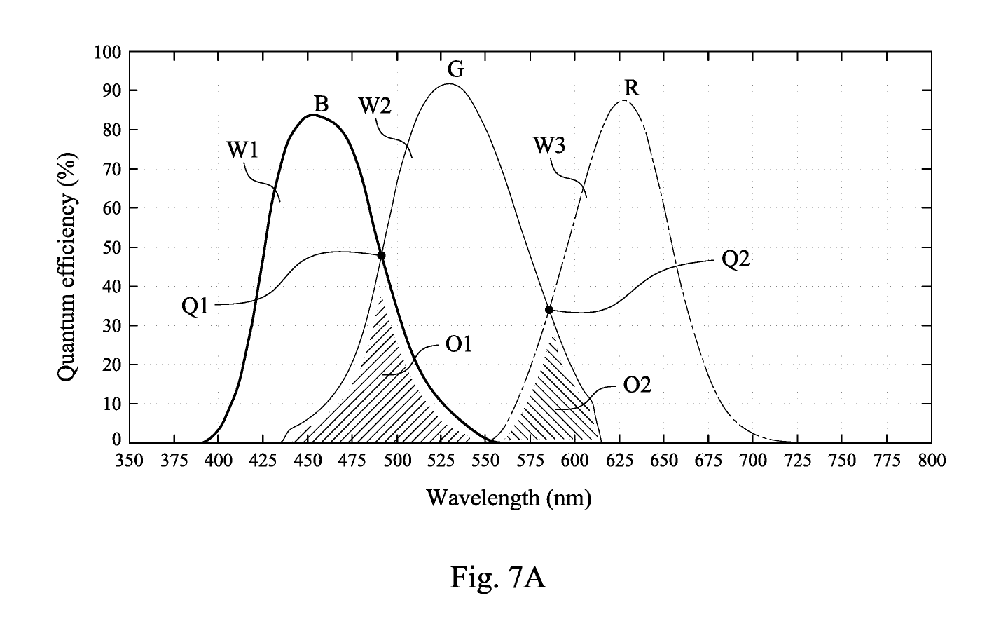

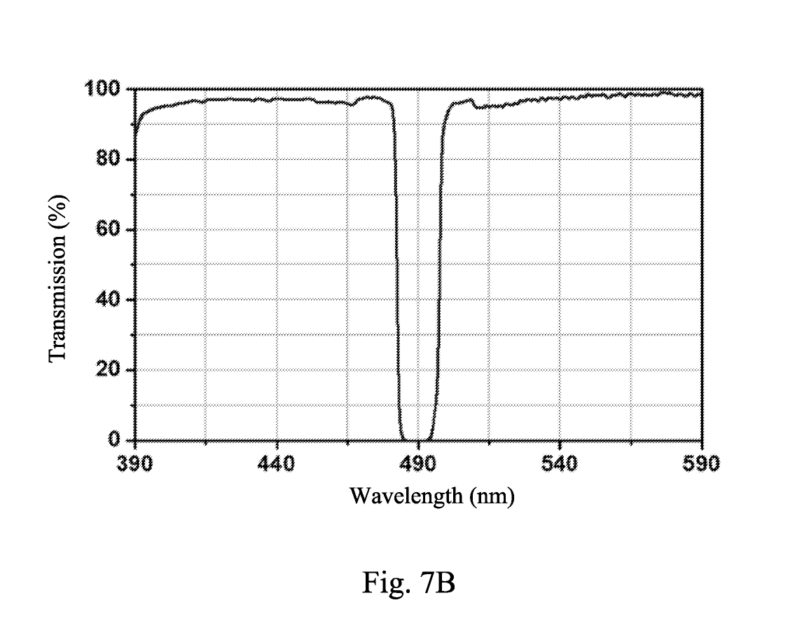

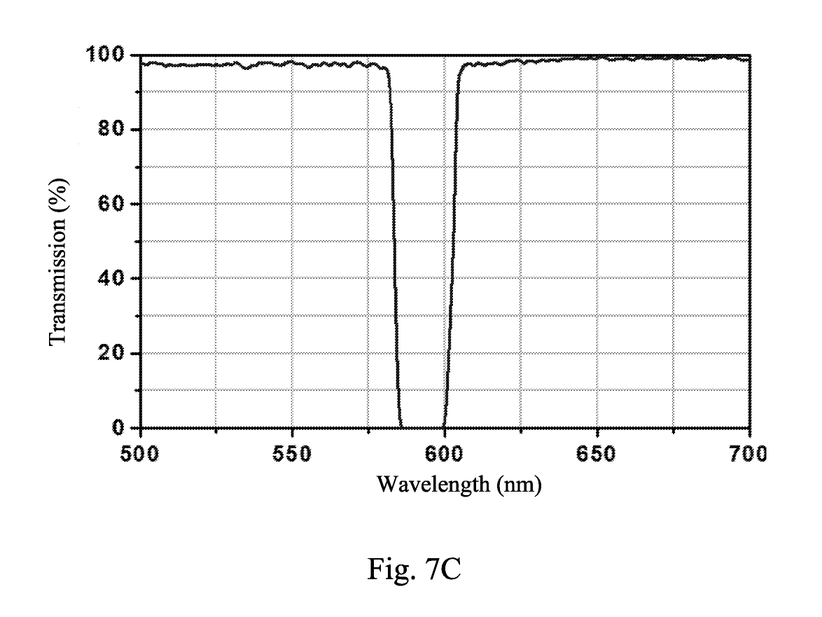

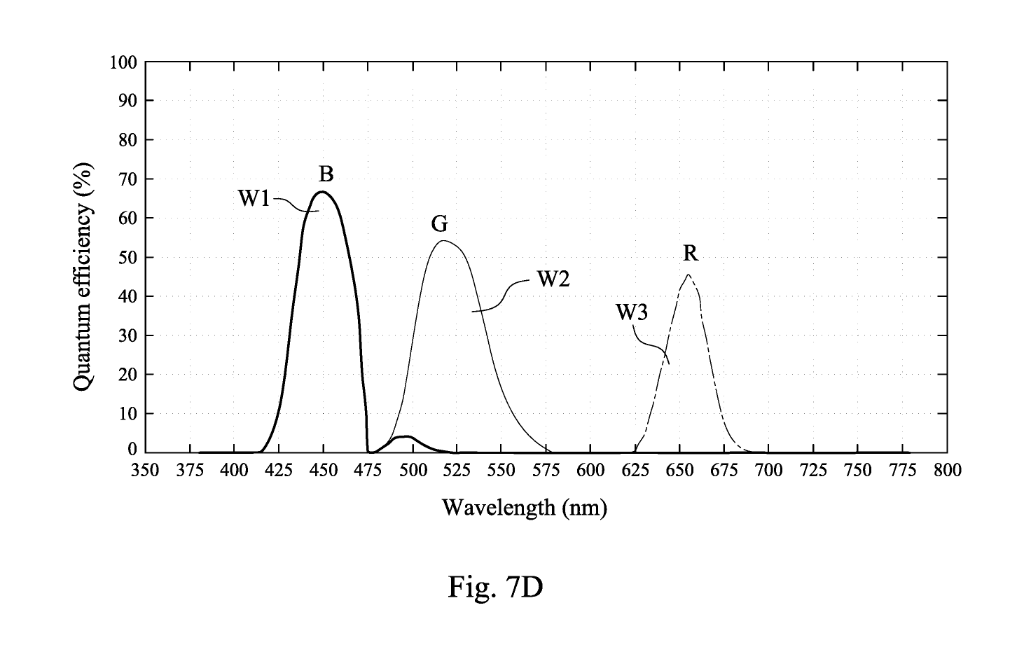

Further referring to FIG. 7A, a drawing shows response curves of an image sensor of a skin analyzer 1 without the configuration of a band-stop filter set. As shown in FIG. 7A, there are two overlapping signals O1 and O2 without the configuration of the band-stop filter set. The overlapping signal O1 occurs between a blue (B) band W1 and a green (G) band W2, and the overlapping signal O2 occurs between a green (G) band W2 and a red (R) band W3. Thus, each of the converted independent components cannot present original features of the facial skin of the subject effectively. The data of FIG. 7A is listed as Table 1:

TABLE 1

| | Center | Full Width at Half |
|---|---|---|
| | Wavelength | Maximum |
| # | (nm) | (nm) |
| W1 | 450 +/− 2 | +/−50 |
| W2 | 530 +/− 2 | +/−50 |
| W3 | 625 +/− 2 | +/−50 |
| O1 | 490 +/− 2 | +/−25 |
| O2 | 590 +/− 2 | +/−25 |

In order to suppress the overlapping signals of the B-G band and the G-R band, the band-stop filter set 206 of the first example can include a first band-stop filter 2062 and a second band-stop filter 2064 as shown in FIG. 2A. Furthermore, a center wavelength of a band-stop of the first band-stop filter 2062 can be set up at an intersection Q1 of the blue band and the green band (B-G band) as shown in FIG. 7A. Moreover, a center wavelength of a band-stop of the second band-stop filter 2064 can be set up at an intersection Q2 of the green band and the red band (G-R band).

The first band-stop filter 2062 is a blue-green filter, and the second band-stop filter is a green-red filter. When an upper limit wavelength of a band-stop of the first band-stop filter 2062 is WL1 and a lower limit wavelength of a band-stop of the second band-stop filter 2064 is WL2, the following condition is satisfied: 70 nm<WL2−WL1<100 nm.

Additionally, the rejected band of the first band-stop filter 2062 is ranging from 471 nm to 504 nm. Furthermore, the rejected band has a center wavelength of 488 nm, with a full width at a half maximum of 15 nm and an error within 2 nm in positive or in negative. Further referring to FIG. 7B, a drawing shows a transmission data of the first band-stop filter 2062 of the skin analyzer 1 according to the first example of the present disclosure. The transmission of the first band-stop filter 2062 between 482 nm and 498 nm is less than 50%. The rejected band of the second band-stop filter 2064 is ranged from 572 nm to 616 nm. Moreover, the rejected band has a center wavelength of 594 nm, a full width at a half maximum of 23 nm and an error within 2 nm in positive or in negative. As shown in FIG. 7C, a drawing shows a transmission data of the second band-stop filter 2064 of the skin analyzer 1 according to the first example of the present disclosure. The transmission of the second band-stop filter 2064 between 583 nm and 603 nm is less than 50%.

Please refer to FIG. 7D. FIG. 7D is a drawing showing response curves of an image sensor of the skin analyzer 1 according to the first example of the present disclosure. The data of FIG. 7D is listed as Table 2:

TABLE 2

| | Center | Full Width at Half |
|---|---|---|
| | Wavelength | Maximum |
| # | (nm) | (nm) |
| W1 | 450 +/− 2 | +/−25 |
| W2 | 520 +/− 2 | +/−25 |
| W3 | 660 +/− 2 | +/−25 |

As shown in FIG. 7D and Table 2, the overlapping signals between the blue band W1 and the green band W2, and the overlapping signals between the green band W2 and the red band W3 are both improved with the band-stop filter set. In addition, a peak quantum efficiency of a red band of the image sensor is less than a peak quantum efficiency of a green band or a blue band of the image sensor. Furthermore, a full width at a half maximum of the band-stop filter set is less than 40 nm.

In addition, a band-pass filter also can be utilized for suppressing the overlapping signals of the B-G band and the G-R band. In particular, a filter with passing bands of 400 nm-471 nm, 504 nm-572 nm and 616 nm-700 nm can be utilized as the band-pass filter as mentioned above.

The flash module 300 also includes a second housing 308. As shown in FIG. 6A, the abovementioned elements of the flash module 300 can be covered by the second housing 308. The second housing 308 is further coupled with the base 100 to prevent elements of the flash module 300 from being affected by the external environmental factors while operating.

In the first example, there is one flash module 300 disposed at each side of the optical imaging system 200, respectively. In addition, the second housing 308 of the flash module 300 is a triangular housing so that the skin analyzer 1 of the first example in the present disclosure has a cat's-face shaped appearance. Furthermore, the flash module 300 can be fixed on the base 100 through the second housing 308 so as to be integrated with the base 100. In details, the flash polarizer 302 is located between the flash lamp 304 and the imaging area A. Furthermore, the flash polarizer 302 and the imaging polarizer 204 can be disposed in a relatively orthogonal orientation with each other to allow the light to pass in a single direction.

Other elements of the skin analyzer 1 in the first example, such as the circuit module 400, the computing module 500 and the display module 600, are mentioned above so that there is no further description herein.

### Second Example

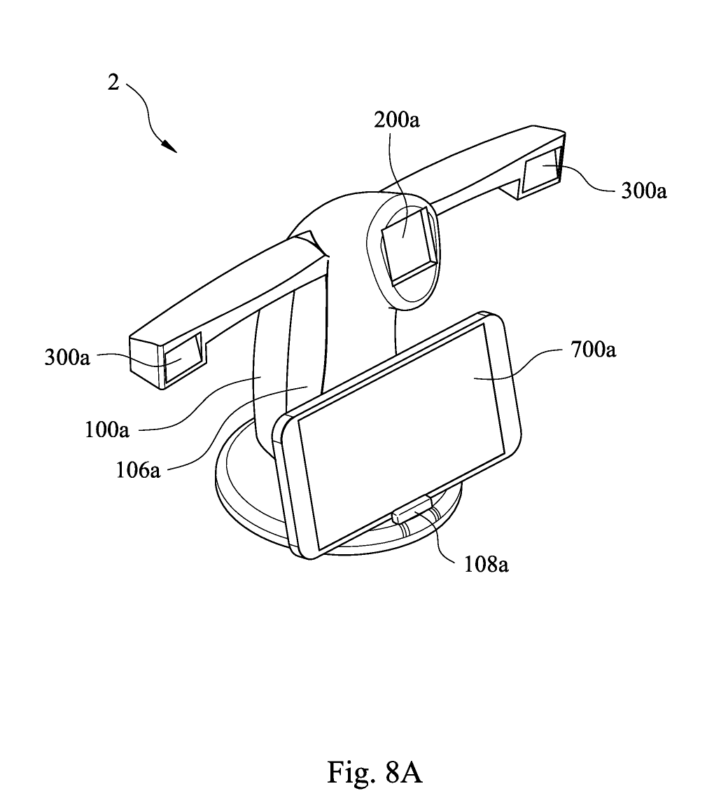

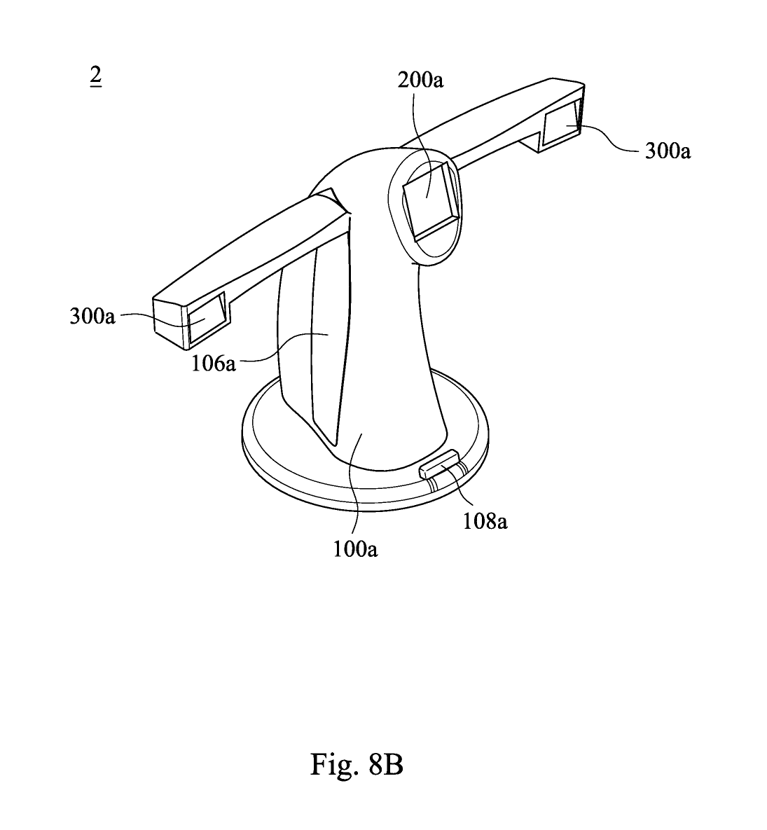

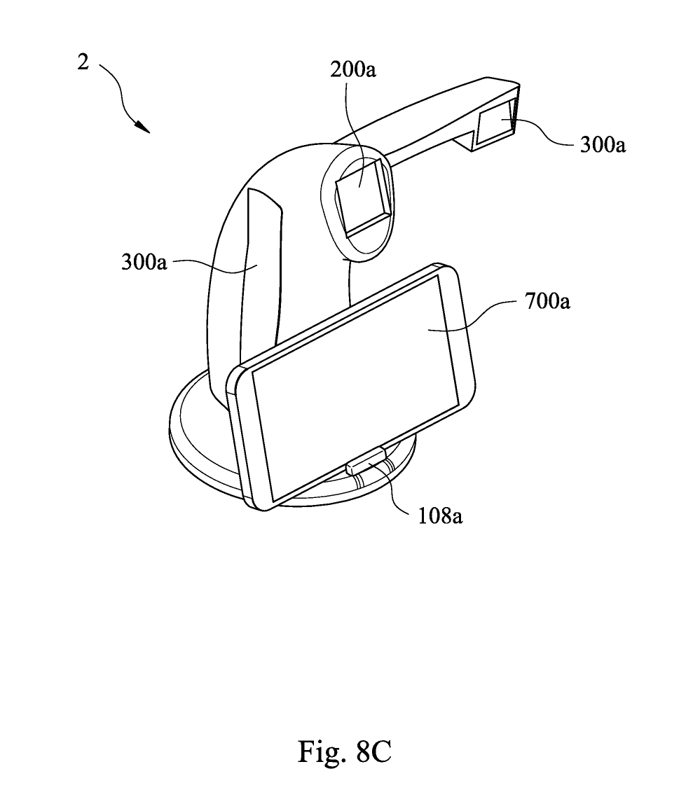

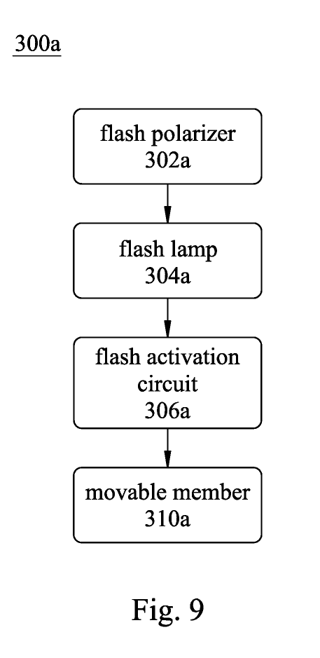

Please refer to FIGS. 8A, 8B, 8C and 9. FIG. 8A is a three-dimensional view of a skin analyzer 2 according to a second example of the present disclosure. FIG. 8B is a three-dimensional view of the skin analyzer 2 of FIG. 8A without the configuration of a portable device 700a. FIG. 8C is a schematic view of a flash module 300a in a folded position of the skin analyzer 2 according to the second example of the present disclosure. FIG. 9 is a structural schematic view of the flash module 300a of the skin analyzer 2 according to the second example of the present disclosure. As shown in FIG. 8A, the skin analyzer 2 of the second example in the present disclosure includes a base 100a, an optical imaging system 200a, two flash modules 300a, a circuit module (not shown), a computing module (not shown) and a display module (not shown). The computing module and the display module of the skin analyzer 2 herein are also built in the portable device 700a. In the second example, the configuration and the pathway of the signal transmission between the optical imaging system 200a, the flash modules 300a, the circuit module, the computing module and the display module are the same as the first example so that a description in this regard will not be provided again herein.

In the second example, the structure of the base 100a of the skin analyzer 2 has a narrow top and a wide bottom so that an additional support stand for the base 100a is not required herein. In addition, a protrusion 108a is formed on the base 100a for fixing the portable device 700a thereon.

The base 100a has two second receiving grooves 106a. Each of the second receiving grooves 106a is disposed at each side of the base 100a corresponding to one of the flash modules 300a. As shown in FIG. 9, the second example is similar to the first example except for the flash modules 300a. Each of the flash modules 300a of the second example can include a movable member 310a (shown in FIG. 9) disposed in the base 100a for moving the flash module 300a to an unfolded position (as shown in FIG. 8A and FIG. 8B) or a folded position (as shown in FIG. 8C). Furthermore, the portable device 700a can be detached as shown in FIG. 8B when the skin analyzer 2 is not used. An operation of a user interface of the portable device 700a is then performed to switch on a flash activation circuit 306a of the flash module 300a. Thus, the movable member 310a drives the flash module 300a to move from the unfolded position to the folded position so as to be received in the second receiving groove 106a.

The conditions of the band-stop filter set utilized in the optical imaging system 200a of the skin analyzer 2 according to the second example of the present disclosure are the same as the first example and will not be described herein.

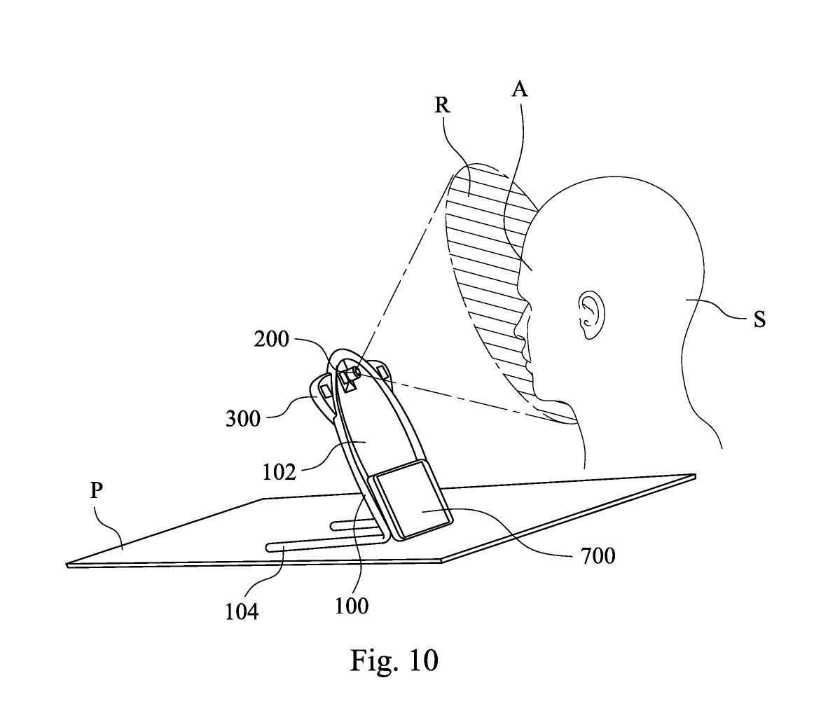

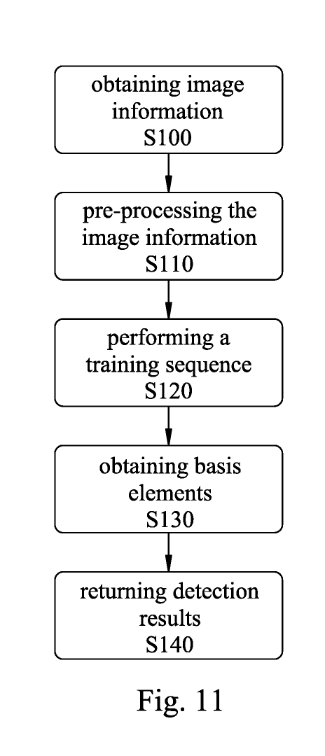

Each element of the skin analyzer 2 in the present disclosure and the connection of these elements thereof have been illustrated as above. Subsequently, details of operating the skin analyzer 1 in the first example and an analysis process are described and shown with FIGS. 10 and 11. FIG. 10 is schematic view showing an operation status of the skin analyzer 1 according to the first example of the present disclosure. FIG. 11 is a flow chart of an image analysis process of the skin analyzer 1 according to the first example of the present disclosure. The image analysis process includes Step S100, Step S110, Step S120, Step S130 and Step S140.

As shown in FIG. 10, the skin analyzer 1 is placed on a surface plane P when a subject S accepts to detect a skin condition of his/her facial skin. An angle between the base 100 and the surface plane P can be adjusted through the stand 104 to allow the facial skin of the subject S (the imaging area A) to be positioned within the field of view of the optical imaging system 200. Then, the user interface of the portable device 700 is operated to allow the display module to send a control signal to the signal synchronization circuit of the circuit module for triggering the flash module 300 and the optical imaging system 200. The image of the imaging area A is captured through the imaging polarizer with the band-stop filter set, and then transmitted to the computing module through the data transmission module of the circuit module.

As shown in FIG. 11, the computing module obtains image information (Step S100) and then pre-processes the image information (Step S110). A training sequence is performed as shown in Step S120. Basis elements, such as an intensity and a distribution of signals of the first independent component and the second independent component, of the skin condition of the subject S are then obtained (Step 130) for evaluating quantitative indicators of the skin condition. An analyzed result will be returned to the display module (Step S140). Finally, the analyzed result and the image information of the imaging area A are shown by the display module.

### Third Example

Please refer to FIGS. 12A, 12B, 12C, 12D and 12E. FIG. 12A is a front view of a skin analyzer 3 according to a third example of the present disclosure. FIG. 12B is a three-dimensional view of the skin analyzer 3 of FIG. 12A. FIG. 12C is a front view of the skin analyzer 3 of FIG. 12A without the configuration of a display module 6000. FIG. 12D is a right-side view of the skin analyzer 3 of FIG. 12A without the configuration of the display module 6000. FIG. 12E is a rear side view of the skin analyzer 3 of FIG. 12A. As shown in FIG. 12A and FIG. 12B, the skin analyzer 3 includes a base 1000, an optical imaging system 2000, at least one flash module 3000, a circuit module (not shown), a computing module (not shown) and a display module 6000.

As shown in FIG. 12C and FIG. 12D, the base 1000 of the skin analyzer 3 is a substantially rectangular parallelepiped and has a long-side length of the skin analyzer Ls and a short-side length of the skin analyzer Ws. In the third example, the long-side length of the skin analyzer Ls is 15.2 cm, and the short-side length of the skin analyzer Ws is 9.8 cm. Furthermore, the base 1000 includes a first housing 1010 and a second housing 1020, and the second housing 1020 can be rotated relatively to the first housing 1010. Each of the first housing 1010 and the second housing 1020 has a receiving space (not shown) respectively for other parts if necessary. For example, the second housing 1020 can be used for accommodating the optical imaging system 2000 and the flash module 3000, so that the operation of each component of the skin analyzer 3 will not be affected by the external environmental factors.

As shown in FIG. 12E, the skin analyzer 3 can further include a foldable stand 7000 connected to a rear side of the base 1000 (the side away from the subject). Corresponding to the foldable stand 7000, the rear side of the base 1000 further includes a standing angle adjusting apparatus 1030 and a receiving groove 1040. In particular, the foldable stand 7000 is connected to the standing angle adjusting apparatus 1030 so that the foldable stand 7000 can be rotated in an angle 81 against the base 1000 or folded into the receiving groove 1040. More particularly, the standing angle adjusting apparatus 1030 can be but not limited to a pivoting mechanism. Therefore, when the foldable stand 7000 is rotated in the angle θ1 against to the base 1000 (in FIG. 9D, the angle θ1 is 55 degrees), the skin analyzer 3 can stand firmly on a flat surface (not shown). When the foldable stand 7000 is folded into the receiving groove 1040, it is favorable for the portability of the skin analyzer 3.

In the third example, the display module 6000 of the skin analyzer 3 is a portable device. Thus, the skin analyzer 3 can further include a portable device bracket 8000 disposed on the front side of the base 1000 (the side facing toward the subject), and the base 1000 can further include a bracket storage apparatus 1050 corresponding to the portable device bracket 8000. When the subject attempts to obtain the image information of the imaging area (such as face) by the display module 6000 (portable device), the portable device bracket 8000 is rotated in a direction toward the subject from the bracket storage apparatus 1050 so that the portable device can be placed on the portable device bracket 8000 (as shown in FIGS. 12A and 12B). Furthermore, the display module 6000 can be detached from the portable device bracket 8000 when the skin analyzer 3 is not in use, and the portable device bracket 8000 can be rotated in a direction away from the subject so that the portable device bracket 8000 can be stored into to the bracket storage apparatus 1050 (as shown in FIG. 12C or FIG. 12D).

In particular, the aforementioned portable device is a substantially rectangular parallelepiped and has a long-side length of the portable device Lm and a short-side length of the portable device Wm, and the long-side length of the portable device Lm is larger than the short-side length of the portable device Wm. More particularly, the long-side length of the portable device Lm and the short-side length of the skin analyzer Ws are disposed in parallel, and the short-side length of the portable device Wm and the long-side length of the skin analyzer Ls are disposed in parallel.

Referring to FIG. 12F, it is a schematic view of a view angle adjusting apparatus 9000 of the skin analyzer 3 of FIG. 12A. As mentioned above, the first housing 1010 and the second housing 1020 are integrated into the base 1000 representing as a substantially rectangular parallelepiped, and the second housing 1020 can be rotated with respect to the first housing 1010. In particular, the skin analyzer 3 can further include a view angle adjusting apparatus 9000 so that the second housing 1020 can be rotated with respect to the first housing 1010, so as to allow the optical imaging system 2000 to rotate upward or downward within an angle 82 (as shown in FIG. 12B). More particularly, as shown in FIG. 12F, the view angle adjusting apparatus 9000 can include a body 9010, two fixed ends 9020, a through-hole 9030 and a pivot axle 9040. The two fixed ends 9020 are respectively extended from two ends of the body 9010, and an extended direction of the fixed end 9020 is perpendicular to an extended direction of the through-hole 9030. The pivot axle 9040 is disposed in the through-hole 9030. Thus, the view angle adjusting apparatus 9000 can be fixed in the first housing 1010 via the two fixed ends 9020, and the second housing 1020 is rotatable connected to the pivot axle 9040 so that the second housing 1020 can be rotated with respect to the first housing 1010.

It must be noted that an engaging member (not shown) can be disposed between the view angle adjusting apparatus 9000 and the second housing 1020 so as to precisely adjust the angle 82 by rotating upward or downward of the optical imaging system 2000. Because the view angle adjusting apparatus 9000 is used for rotating the optical imaging system 2000 upward or downward so as to obtain accurate image information of the imaging area, apparatuses which can facilitate the rotation of the second housing 1020 with respect to the first housing 1010 and in turn rotate the optical imaging system 2000 are suitable of being the view angle adjusting apparatus 9000 of the present invention, so that the detailed information of the view angle adjusting apparatus 9000 are not be described herein. In addition, as shown in FIG. 12F, two of the view angle adjusting apparatus 9000 are disposed on the right side and the left side of the second housing 1020 respectively, and the present disclosure is not limited thereto.

Although it is not shown in FIG. 12A to FIG. 12E, the detailed structure of the flash module 3000 according to the third example is substantially the same as the flash module 30 shown in FIG. 3. Furthermore, as shown in FIG. 12A, the flash module 3000 includes an imaging polarizer 3020, a flash lamp 3400 and a flash activation circuit (not shown), and the flash polarizer 3020 can be disposed on an external surface of the flash lamp 3400 but not limited thereto. In addition, there can be two of the flash module 3000 of the skin analyzer 3, and the two flash modules 3000 are disposed at two sides of the optical imaging system, respectively. More particularly, when a distance between the two flash modules Df is 2.16 cm, the short-side length of the skin analyzer Ws is 9.8 cm, and the value of Df/Ws is 0.22. Furthermore, the flash polarizer 3020 and the imaging polarizer (not shown) can be disposed in a relatively orthogonal orientation with each other to allow the light to pass in a single direction in the third example.

In addition, the detailed structure of the optical imaging system 2000 according to the third example of the present disclosure is substantially the same as the optical imaging system 20 shown in FIG. 4A to FIG. 4D. The conditions of the band-stop filter set utilized in the optical imaging system 2000 of the skin analyzer 3 according to the third example of the present disclosure are the same as the first example. The circuit module and the computing module of the skin analyzer 3 according to the third example of the present disclosure are also substantially the same as the above description so they won't be described again.

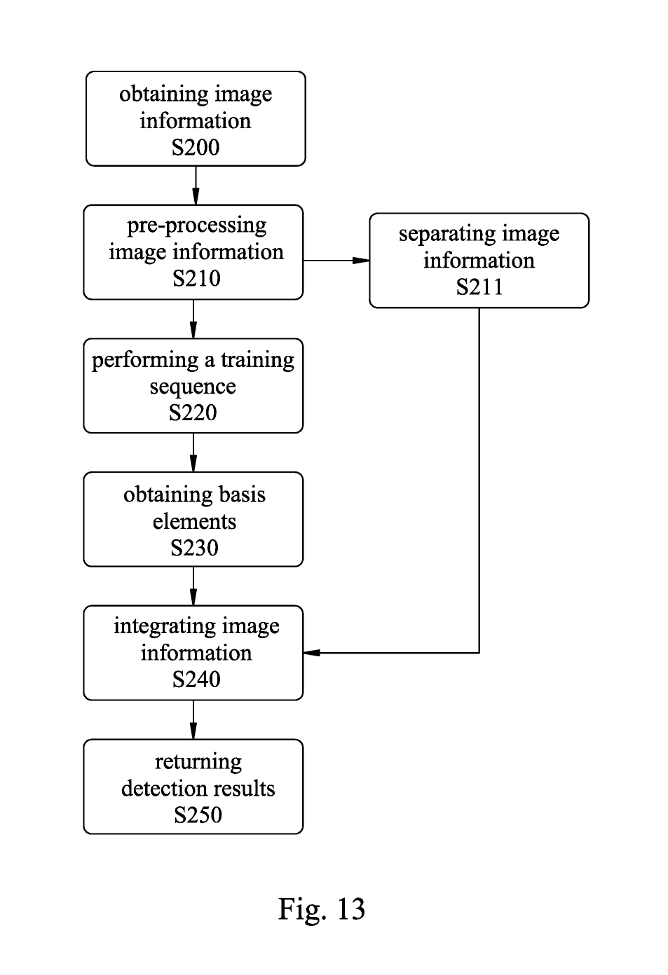

Subsequently, details of operating the skin analyzer 3 in the third example and an analysis process are described and shown with FIG. 13. FIG. 13 is a flow chart of an image analysis process of the skin analyzer 3 according to the third example of the present disclosure. The image analysis process includes Step S200, Step S210, Step S211, Step S220, Step S230, Step S240 and Step S250.

Step S200 is for obtaining the image information. In detail, the skin analyzer 3 is placed on a flat surface when the subject attempts to analyze the skin condition of his/her facial skin. A view angle of the optical imaging system 2000 can be adjusted via the foldable stand 7000 and the view angle adjusting apparatus 9000 so as to allow the facial skin of the subject (the imaging area A) to be positioned within the view angle of the optical imaging system 2000. Then, the image of the facial skin of the subject can be viewed directly via the display module 6000. Next, the subject photographs at least one imaging areas through the user interface of the portable device 7000 and the display module to trigger the flash module 3000 and the optical imaging system 2000 after sending a control signal to the signal synchronization circuit of the circuit module. Then, as shown in Step S200, the image information of the imaging area A is captured with the use of the imaging polarizer and the band-stop filter set, and then the image information of the imaging area A is sent to the computing module via the data transmission module of the circuit module.

Step S210 is for pre-processing the image information. In particular, the computing module will preprocess the image information after obtaining the image information. For example, the aforementioned image information will undergo a lens shading correction so as to obtain corrected image information. Furthermore, when Step S210 is performed, the image information of facial moles can be recognized and further separated by the threshold definition, which is done in Step S211.

Thereafter, a transformation matrix is generated by the independent component analysis so as to perform a training sequence in Step S220. Basis elements of the skin condition of the subject are obtained in Step S230. The basis elements include a parameter image of hemoglobin (that is, original distribution diagram of the hemoglobin), a first parameter image of melanin (that is, original distribution diagram of melanin) and a controlled parameter image.

Step 240 is for integrating image information. In detail, in Step S240, the image information of facial moles separated in Step S211 is deducted by the first parameter image of melanin obtained in Step S230 so as to obtain a second parameter image of the melanin (that is, a post-corrected distribution diagram of melanin). Finally, in Step S250, the image information obtained in Step S200 and corrected results (the parameter image of hemoglobin obtained in Step S230 and the second parameter image of the melanin obtained in Step S240) will be transmitted to the display module. Thus, the aforementioned corrected results will be analyzed by analysis programs built in the display module (the display module is a portable device in the third example), and the image information and parameter images obtained by the aforementioned steps can be stored, transmitted or shown to professional personnel for diagnosis. Therefore, the flexible usage of the skin analyzer according to the present disclosure can be enhanced.

### Fourth Example

Please refer to FIG. 14, which is a front side view of a skin analyzer 4 according to a fourth example of the present disclosure. In the fourth example, the configuration is substantially the same as the skin analyzer 3 shown in FIG. 3. The skin analyzer 4 includes a base 1100, an optical imaging system 2100, at least one flash module 3100, a circuit module (not shown), a computing module (not shown) and a display module 6100.

In short, the base 1100 of the skin analyzer 4 is also a substantially rectangular parallelepiped and has a long-side length of the skin analyzer and a short-side length of the skin analyzer. Moreover, the base 1100 also includes a first housing 1110 and a second housing 1120, and each of the first housing 1110 and the second housing 1120 has a receiving space (not shown) respectively for other parts if necessary. For example, the second housing 1120 can be used for accommodating the optical imaging system 2100 and the flash module 3100, so that the operation of each component of the skin analyzer will not be affected by the external environmental factors. Similarly, the skin analyzer 4 includes a portable device bracket 8100 disposed on the front side (the side facing toward the subject) of the base 1100 so that the portable device can be placed on the portable device bracket 8100. Furthermore, the skin analyzer 4 according to the fourth example includes at least one view angle adjusting apparatus 9100 so that the second housing 1120 can be rotated with respect to the first housing 1110 so as to drive the optical imaging system 2100 to rotate upward or downward within a certain angle range. As for other elements of the skin analyzer 4, such as the foldable stand, the standing angle adjusting apparatus and the bracket storage apparatus are substantially the same as the third example.

However, the flash module 3100 of the fourth example is a full band flash module, which is different from the flash module 3000 of the third example. Therefore, the flash polarizer of the skin analyzer can be omitted. In detail, the flash module 3100 includes a red flash lamp 3110, a green flash lamp 3120, a blue flash lamp 3130 and a flash activation circuit (not shown), and the configuration of the optical imaging system 2100 is the same as the optical imaging system 20A shown in FIG. 4D, which includes only the imaging module and the conditions of the band-stop filter set utilized in the optical imaging system 2100 of the skin analyzer 4 according to the fourth example of the present disclosure are the same as the first example and not be described herein.

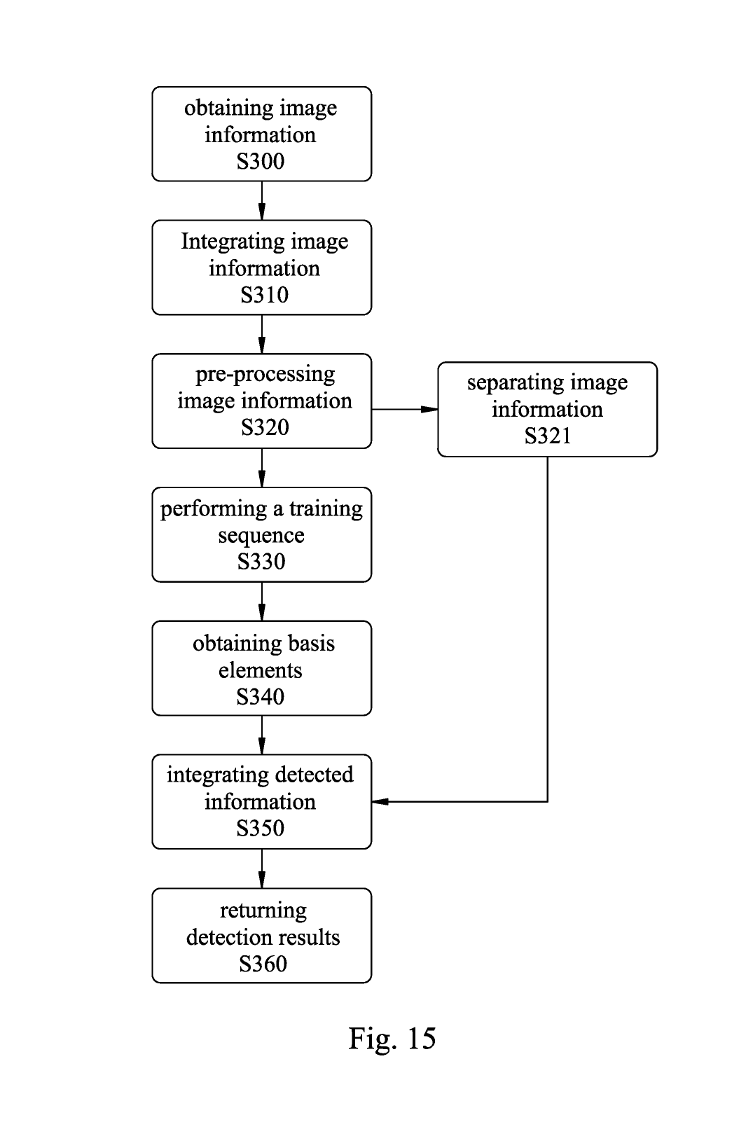

Subsequently, details of an analysis method by operating the skin analyzer 4 in the fourth example are described and shown with FIG. 15. FIG. 15 is a flow chart of an image analysis process of the skin analyzer 4 according to the fourth example of the present disclosure. The image analysis process includes Step S300, Step S310, Step S320, Step S321, Step S330, Step S340, Step S350, and Step S360.

Step S300 is for obtaining the image information. In the fourth example, unlike the process shown in the third example, at least three imaging areas of the subject are captured by the skin analyzer 4 so as to obtain three sets of image information. The three image information sets are sent to the computing module through the data transmission module of the circuit module. In Step 310, these image information sets (obtained in Step S300) will be processed by the computing module so as to generate integrated image information. In detail, the aforementioned imaging areas can be areas of the left cheek, the front face and the right cheek of the subject. When the three imaging areas are photographed by the skin analyzer, there should be an overlapping section between every two imaging areas so as to correct reflections on parts of the facial skin of the subject. Then, image information without the reflections from one of the two imaging areas is selected, and the three image information sets can be connected so as to obtain integrated image information.

Then, Step S320 is for pre-processing the image information. Particularly, the computing module will preprocess the aforementioned integrated image information. For example, the aforementioned integrated image information will undergo a lens shading correction so as to obtain corrected image information. Furthermore, when Step S320 is performed, the image information of facial moles can be identified and further separated by the threshold definition, which is done in Step S321.

Thereafter, a transformation matrix is generated by the independent component analysis so as to perform a training sequence in Step S330. Basis elements of the skin condition of the subject are obtained in Step S340. The basis elements include a parameter image of hemoglobin (that is, original distribution diagram of the hemoglobin), a first parameter image of melanin (that is, original distribution diagram of melanin) and a control parameter image.

Step 350 is for integrating image information. In detail, in Step S350, the image information of facial moles separated in Step S321 is deducted by the first parameter image of melanin obtained in Step S340 so as to obtain a second parameter image of the melanin (that is, a post-corrected distribution diagram of melanin). Furthermore, in Step S360, the three image information sets obtained in Step S300 and corrected results (the parameter image of hemoglobin obtained in Step S340 and the second parameter image of the melanin obtained in Step S350) will be returned to the display module. In this time, the aforementioned corrected results will be analyzed by analysis programs built in the display module (the display module is a portable device in the third example), and the image information and the corrected results obtained by the aforementioned steps can be stored, transmitted or shown to professional personnel for further diagnosis.

In conclusion, the present disclosure utilizes the configuration of the band-stop filter set or the band-pass filter to suppress the overlapping signals of the B-G band and the G-R band. Thereby, the features of the image will not be hindered by the overlapping signals so as to improve the quality of the image after the conversion. Furthermore, the skin analyzer of the present disclosure is miniaturized and favorable for carrying on the go. The skin analyzer of the present disclosure can be used along with the existing portable device (such as a portable device or a tablet PC). It is favorable for providing an intuitive and easy-to-operate platform for the subject in accord with common practices of the subject.

Although the present disclosure has been described in considerable detail with reference to certain embodiments thereof, there are other possible embodiments with different parameters. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this to disclosure provided they fall within the scope of the following claims.

What is claimed is:

1. A skin analyzer, comprising:
a base;
an optical imaging system disposed on the base and comprising an imaging polarizer, a band-stop filter set and an imaging module;
at least one flash module disposed on at least one side of the optical imaging system, wherein the flash module comprises a flash polarizer, a flash lamp and a flash activation circuit;
a circuit module disposed in the base and electrically connected with the optical imaging system and the flash module, wherein the circuit module comprises a power control circuit, a data transmission circuit and a signal synchronization circuit;
a computing module having a signal transmitting connection with the circuit module; and
a display module having a signal transmitting connection with the computing module;
wherein the skin analyzer is a substantially rectangular parallelepiped and has a long-side length of the skin analyzer and a short-side length of the skin analyzer, the long-side length of the skin analyzer is Ls, the short-side length of the skin analyzer is Ws, and the following condition is satisfied:
0 cm<Ws<Ls<30 cm.

2. The skin analyzer of claim 1, wherein the skin analyzer further comprises a foldable stand.

3. The skin analyzer of claim 2, wherein the base comprises a standing angle adjusting apparatus, which is connected with the foldable stand.

4. The skin analyzer of claim 1, wherein the display module is a portable device, the skin analyzer further comprises a portable device bracket disposed on a side of the skin analyzer facing a subject, and the portable device is disposed in the portable device bracket.

5. The skin analyzer of claim 4, wherein the base comprises a bracket storage apparatus.

6. The skin analyzer of claim 4, wherein the portable device is a substantially rectangular parallelepiped and has a long-side length of the portable device and a short-side length of the portable device, the long-side length of the portable device and the short-side length of the skin analyzer are disposed in parallel, and the short-side length of the portable device and the long-side length of the skin analyzer are disposed in parallel.

7. The skin analyzer of claim 1, wherein the skin analyzer further comprises a view angle adjusting apparatus.

8. The skin analyzer of claim 7, wherein the view angle adjusting apparatus is capable of rotating the optical imaging system in an angle less than or equal to 45 degrees in an upward or downward direction.

9. The skin analyzer of claim 1, wherein there are two flash modules which are disposed at two sides of the optical imaging system, respectively.

10. The skin analyzer of claim 9, wherein a distance between the two flash modules is Df, the short-side length of the skin analyzer is Ws, and the following condition is satisfied:
0.1<Df/Ws<0.7.

11. The skin analyzer of claim 1, wherein the long-side length of the skin analyzer is Ls, and the following condition is satisfied:
5 cm<Ls<20 cm.

12. The skin analyzer of claim 1, wherein the imaging module comprises an imaging lens assembly and an image sensor, and a peak quantum efficiency of a red band of the image sensor is less than a peak quantum efficiency of a green band or a blue band of the image sensor.

13. The skin analyzer of claim 1, wherein a full width at a half maximum of a filter region of the band-stop filter set is less than 40 nm.

14. The skin analyzer of claim 1, wherein the band-stop filter set comprises a first band-stop filter and a second band-stop filter; the first band-stop filter is a blue-green filter, the second band-stop filter is a green-red filter, an upper limit wavelength of a band-stop of the first band-stop filter is WL1, and a lower limit wavelength of a band-stop of the second band-stop filter is WL2, the following condition is satisfied:
70 nm<WL2−WL1<100 nm.

15. A skin analyzer, comprising:
a base;
an optical imaging system disposed on the base and comprising an imaging module;
at least one flash module disposed on at least one side of the optical imaging system, wherein the flash module comprises a red-light flash, a green-light flash, a blue-light flash and a flash activation circuit;
a circuit module disposed in the base and electrically connected with the optical imaging system and the flash module, wherein the circuit module comprises a power control circuit, a data transmission circuit and a signal synchronization circuit;
a computing module having a signal transmitting connection with the circuit module; and
a display module having a signal transmitting connection with the computing module;
wherein the skin analyzer is a substantially rectangular parallelepiped and has a long-side length of the skin analyzer and a short-side length of the skin analyzer, the long-side length of the skin analyzer is Ls, the short-side length of the skin analyzer is Ws, and the following condition is satisfied:
0<Ws<Ls<30 cm.

16. The skin analyzer of claim 15, further comprising:
a foldable stand.

17. The skin analyzer of claim 15, wherein the display module is a portable device, the skin analyzer further comprises a portable device bracket disposed on a side of the skin analyzer facing toward a subject, and the portable device is disposed in the portable device bracket of the portable device.

18. The skin analyzer of claim 15, further comprising:
a view angle adjusting apparatus.

19. The skin analyzer of claim 15, wherein the long-side length of the skin analyzer is Ls, and the following condition is satisfied:
5 cm<Ls<20 cm.

20. The skin analyzer of claim 15, wherein the optical imaging system comprises a band-stop filter set.

21. The skin analyzer of claim 20, wherein the imaging module comprises an imaging lens assembly and an image sensor, and a peak quantum efficiency of a red band of the image sensor is less than a peak quantum efficiency of a green band or a blue band of the image sensor.

22. The skin analyzer of claim 20, wherein a full width at a half maximum of a filter region of the band-stop filter set is less than 40 nm.

23. The skin analyzer of claim 20, wherein the band-stop filter set comprises a first band-stop filter and a second band-stop filter, the first band-stop filter is a blue-green filter, the second band-stop filter is a green-red filter, an upper limit wavelength of a band-stop of the first band-stop filter is WL1, a lower limit wavelength of a band-stop of the second band-stop filter is WL2, and the following condition is satisfied:
70 nm<WL2−WL1<100 nm.
